SEQUENCE LISTING (1 sequence)

SEQ ID NO: 1 - protein, 29 aa, Artificial Sequence
  synthetic peptide of GHRH
YADAIFTNSYRKVLGQLSARKLLQDIMSR

(19) United States
(12) Patent Application Publication
(10) Pub. No.: US 2015/0166617 A1
(43) Pub. Date: Jun. 18, 2015

(54) COMPOSITIONS AND METHODS OF TREATING ALZHEIMER'S DISEASE

(71) Applicants: UNIVERSITY OF MIAMI, Miami, FL (US); UNITED STATES OF AMERICA, REPRESENTED BY THE DEPARTMENT OF VETERANS AFFAIRS, Washington, DC (US)

(72) Inventors: Andrew V. Schally, Miami, FL (US); Miklos Jasberenyi, Miami, FL (US); Norman L. Block, Maimi, FL (US)

(21) Appl. No.: 14/409,259

(22) PCT Filed: Jun. 27, 2013
(86) PCT No.: PCT/US13/48381

§ 371 Date: Dec. 18, 2014

Related U.S. Application Data
(60) Provisional application No. 61/664,860, filed on Jun. 27, 2012.

(51) Int. Cl. C07K 14/47; A61K 45/06; A61K 38/17

(52) U.S. Cl. CPC C07K 14/47; A61K 38/1709; A61K 45/06

(57) ABSTRACT

Disclosed herein are compositions of GHRH peptide antagonists, and methods to treat Alzheimer's disease and other neurodegenerative disorders. Such compounds are useful for therapeutics, for protecting neuronal cells from cell-death and for promoting neuronal cell viability.

### CROSS-REFERENCE TO RELATED APPLICATIONS

This application is a U.S. national stage filing under 35 U.S.C. §371 of International Application No. PCT/US20130/48381 filed Jun. 27, 2013 entitled “Compositions and Methods of Treating Alzheimer's Disease”, which is incorporated herein by reference in its entirety, which in turn claims priority to the Provisional Application No. 61/664,860 filed on Jun. 27, 2012.

### GOVERNMENT INTERESTS

This work was supported by the Medical Research Service of the Veterans Affairs Department, University of Miami, Miller School of Medicine, Departments of Pathology and Medicine, Division of Hematology/Oncology, the South Florida Veterans Affairs Foundation for Research and Education, and the L. Austin Weeks Endowment for Urologic Research. This work was also supported in part by a grant from the AUA Foundation Research Scholars Program and the AUA Southeastern Section.

### PARTIES TO A JOINT RESEARCH AGREEMENT

Not Applicable

### INCORPORATION OF SEQUENCE LISTING

A computer readable form of the sequence listing “Biscayne 201 Seq Listing_ST25.txt” (750 bytes) submitted via EFS-WEB and created on Dec. 16, 2014, is herein submitted.

### BACKGROUND

Not Applicable

### BRIEF SUMMARY OF THE INVENTION

The present disclosure is related to compositions and methods for treating Alzheimer's disease and other neurodegenerative disorders. In one embodiment, a method of treating a subject having Alzheimer's disease may involve administering to the subject a therapeutic amount of growth hormone releasing hormone (GHRH) peptide antagonist having amino acid sequences of formula I. In some embodiments, the formula I is as follows:

R¹-Tyr¹-D-Arg²-Asp³-A⁴-Ile⁵-A⁶-Thr⁷-A⁸-Har⁹-A¹⁰-A¹¹-A¹²-Val¹³-Leu¹⁴-A¹⁵-Gln¹⁶-A¹⁷-Ser¹⁸-Ala¹⁹-A²⁰-A²¹-Leu²²-Leu²³-Gln²⁴-Asp²⁵-Ile²⁶-Nle²⁷-D-Arg²⁸-A²⁹-R²-R³-NH₂,
wherein R¹ is PhAc, Nac, Oct, N-Me-Aib, Dca, Ac-Ada, Fer, Ac-Amc, Me-NH-Sub, PhAc-Ada, Ac-Ada-D-Phe, Ac-Ada-Phe, Dca-Ada, Nac, Nac-Ada, Ada-Ada, or CH₃(CH₂)₁₀-CO-Ada;
A⁴ is Ala or Me-Ala;
A⁶ is Cpa or Phe(F)₅;
A⁸ is Ala, Pal, Dip, or Me-Ala;
A¹⁰ is FPa5,Tyr(Alk) where Alk is Me or Et;
A¹¹ is His or Arg;
A¹² is Lys, Lys(0-11), Lys(Me)₂, or Orn;
A¹⁵ is Abu or Orn;
A¹⁷ is Leu or Glu;
A²⁰ is Har or His;
A²¹ is Lys, Lys(Me)₂ or Orn;
A²⁹ is Har, Arg or Agm;
R² is β-Ala, Amc, Apa, Ada, AE₂A, AE₄P, ε-Lys(α-NH₂), Agm, or absent;

and
R³ is Lys(Oct), Ahx, or absent.
   In some embodiments, the GHRH peptide antagonist has amino acid sequences of formula II as described further herein.

In a further embodiment, a method for treating a subject having amyloid plaque deposits may involve administering to the subject a therapeutic amount of growth hormone releasing hormone (GHRH) peptide antagonist having amino acid sequences of formula I. In some embodiments, the GHRH peptide antagonist has amino acid sequences of formula II as described further herein.

In another embodiment, a method of treating a subject having a neurodegenerative disease may involve administering to the subject a therapeutic amount of growth hormone releasing hormone (GHRH) peptide antagonist having amino acid sequences of formula I. In some embodiments, the GHRH peptide antagonist has amino acid sequences of formula II as described further herein. The neurodegenerative diseases may be Alzheimer's disease, senile dementia, dementia with Lewy Bodies, Parkinson's disease, Huntington's disease, amyotrophic lateral sclerosis, and combination thereof. In some embodiments, the neurodegenerative diseases may be Mediterranean fever, Muckle-Wells syndrome, idiopathic myeloma, amyloid polyneuropathy, amyloid cardiomyopathy, systemic neuritic amyloidosis, amyloid polyneuropathy, hereditary cerebral hemorrhage with amyloidosis, Down's syndrome, Scrapie, Creutzfeldt-Jacob disease, Kuru, Gerstamnn-Straussler-Scheinker syndrome, medullary carcinoma of the thyroid, isolated atrial amyloid, β2-microglobulin amyloid in dialysis patients, inclusion body myositis, β2-amyloid deposits in muscle wasting disease, type II diabetes, and combinations thereof.

In an additional embodiment, a method of protecting neuronal cells from oxidative stress may include contacting the neuronal cells with a growth hormone releasing hormone (GHRH) peptide antagonist having amino acid sequences of formula I. In some embodiments, the GHRH peptide antagonist has amino acid sequences of formula II as described further herein.

In a further embodiment, a method of improving viability of neuronal cells may include contacting the neuronal cells with a growth hormone releasing hormone (GHRH) peptide antagonist having amino acid sequences of formula I. In some embodiments, the GHRH peptide antagonist has amino acid sequences of formula II as described further herein.

### DESCRIPTION OF DRAWINGS

FIG. 1 illustrates the effect of the GHRH antagonist, MIA-690, on the progressive changes of the behavioral parameters of the 5XFAD transgenic mice in Morris water maze (MWM) experiments. Mice were treated with daily subcutaneous injections of GHRH antagonist MIA-690 at doses of 2, 5, and 10 μg for 6 months. The pooled standard errors (PSE)s of the groups were the following (A) control: 36.0, MIA-690 (2 μg): 41.2, MIA-690 (5 μg): 33.3, MIA-690 (10 μg): 36.9; (B) control: 21.8, MIA-690 (2 μg): 31.4, MIA-690 (5 μg): 20.6, MIA-690 (10 μg): 16.0; (C) control: 53.1, MIA-690 (2 μg): 63.4, MIA-690 (5 μg): 51.9, MIA-690 (10 μg): 38.0; (D) control: 43.6, MIA-690 (2 μg): 74.6, MIA-690 (5 μg): 45.3, MIA-690 (10 μg): 40.0. *=p<0.05 vs. control according to repeated measure general linear model analysis.

FIG. 2 shows the effect of MIA-690 on the progressive changes of the probe parameters of the 5XFAD transgenic mice in Morris water maze (MWM) experiments. Mice were treated with daily subcutaneous injections of GHRH antagonist MIA-690 at doses of 2, 5, and 10 μg for 6 months. Data are represented as mean±SEM.

FIG. 3 shows the effect of MIA-690 on the behavioral parameters of the 5XFAD transgenic mice in the spatial acquisition of the MWM experiments, during the 1^{st} and 6^{th} month. Mice were treated with daily subcutaneous injections of GHRH antagonist MIA-690 at doses of 2, 5, and 10 μg for 6 months. The pooled standard errors (PSE)s of the groups were the following (A) control: 28.32, MIA-690 (2 μg): 44.1, MIA-690 (5 μg): 25.5, MIA-690 (10 μg): 37.6; (B) control: 30.8, MIA-690 (2 μg): 47.4, MIA-690 (5 μg): 25.0, MIA-690 (10 μg): 43.2; (C) control: 20.1, MIA-690 (2 μg): 15.9, MIA-690 (5 μg): 17.6, MIA-690 (10 μg): 28.6; (D) control: 22.0, MIA-690 (2 μg): 16.6, MIA-690 (5 μg): 19.8, MIA-690 (10 μg): 23.5. *=p<0.05 vs. control according to repeated measure general linear model analysis.

FIG. 4 demonstrates the effect of MIA-690 on the survival of the 5XFAD transgenic mice over 6 months. Numbers on each line represent the estimated mean survival time for each group. Mice were treated with daily subcutaneous injections of GHRH antagonist MIA-690 at doses of 2, 5, and 10 μg for 6 months.

FIG. 5 shows the effect of MIA-690 on the accumulation of amyloid-β₁-42 and T-protein in the brain of 5XFAD transgenic mice. Mice were treated with daily subcutaneous injections of GHRH antagonist MIA-690 at doses of 2, 5, and 10 μg for 6 months. (*=p<0.05 vs. control. Data are represented as mean+/−SEM).

FIG. 6 shows the effect of MIA-690 on HCN-2 cells, regarding viability, free radical formation, enzyme and mediator expression in vitro. Cells were treated with 10 μM amyloid-β₁₋₄₂, and the combination treatments with 10 μM amyloid-β₁₋₄₂ and the 3 doses (10 nM, 100 nM and 1 μM) of MIA-690. Abbreviations: ROS: reactive oxygen species, GPx: glutathione-peroxidase, BDNF: brain derived neurotrophic factor. *=p<0.05 vs. control. Data are represented as mean+/−SEM.

FIG. 7 demonstrates the effect of the GHRH antagonist peptides (MIA-602, MIA-606 and MIA-640) on the viability of HCN-2 cells treated by amyloid-β₁₋₄₂. Differentiated HCN-2A neuronal cells were treated with 10 μM amyloid-β1-42 alone and in combination with 10 nM, 100 nM and 1 μM of MIA-602, MIA-606 and MIA 640. After the treatment, the cell viability was determinate as indicated by the supplier using the Promega Cell Titer 96® Aqueous Non-Radioactive Cell Proliferation Assay. The absorbance level was determined at 490 nm. Data are percentage of control (n=3).

### DETAILED DESCRIPTION

This invention is not limited to the particular processes, compositions, or methodologies described, as these may vary. The terminology used in the description is for the purpose of describing the particular versions or embodiments only, and is not intended to limit the scope of the present invention. Unless defined otherwise, all technical and scientific terms used herein have the same meanings as commonly understood by one of ordinary skill in the art. All publications mentioned herein are incorporated by reference in their entirety. Nothing herein is to be construed as an admission that the invention is not entitled to antedate such disclosure by virtue of prior invention.

As used herein and in the appended claims, the singular forms “a”, “an”, and “the” include plural reference unless the context clearly dictates otherwise. Thus, for example, reference to an “antioxidant” is a reference to one or more antioxidants and equivalents thereof known to those skilled in the art, and so forth.

As used herein, the term “about” means plus or minus 10% of the numerical value of the number with which it is being used. Therefore, about 50% means in the range of 45%-55%.

“Administering” when used in conjunction with a therapeutic means to administer a therapeutic to a patient whereby the therapeutic positively impacts the tissue to which it is targeted. The peptides/compounds described herein can be administered either alone or in combination (concurrently or serially) with other pharmaceuticals. For example, the peptides/compounds can be administered in combination with other anti-cancer or anti-neoplastic agents, or in combination with other cancer therapies other than chemotherapy, such as, for example, surgery or radiotherapy. In some embodiments, the peptides/compounds described herein can also be administered in combination with (i.e., as a combined formulation or as separate formulations) with antibiotics.

The term “animal,” “patient,” or “subject” as used herein includes, but is not limited to, humans and non-human vertebrates such as wild, domestic and farm animals. Preferably, the term refers to humans.

By “pharmaceutically acceptable”, it is meant the carrier, diluent or excipient must be compatible with the other ingredients of the formulation and not deleterious to the recipient thereof.

As used herein, the term “therapeutic” means an agent utilized to discourage, combat, ameliorate, prevent or improve an unwanted condition, disease or symptom of a patient.

A “therapeutically effective amount” or “effective amount” of a composition is a predetermined amount calculated to achieve the desired effect, i.e., to ameliorate, prevent or improve an unwanted condition, disease or symptom of a patient. The activity contemplated by the present methods includes both therapeutic and/or prophylactic treatment, as appropriate. The specific dose of the peptides/compounds or the peptides administered according to this invention to obtain therapeutic and/or prophylactic effects will, of course, be determined by the particular circumstances surrounding the case, including, for example, the peptides/compounds administered, the route of administration, and the condition being treated. The effective amount administered may be determined by a physician in the light of the relevant circumstances including the condition to be treated, the choice of peptides/compounds to be administered, and the chosen route of administration. A therapeutically effective amount of the peptide/compound of this invention is typically an amount such that when it is administered in a physiologically tolerable excipient composition, it is sufficient to achieve an effective systemic concentration or local concentration in the target tissue.

Generally speaking, the term “tissue” refers to any aggregation of similarly specialized cells which are united in the performance of a particular function.

As used herein, “analog” of polypeptides refers to an amino acid sequence that is altered by one or more amino acid residues. The analog may have “conservative” changes, wherein a substituted amino acid has similar structural or chemical properties (e.g., replacement of leucine with isoleucine). More rarely, an analog may have “nonconservative” changes (e.g., replacement of glycine with tryptophan). Analogous minor variations may also include amino acid deletions or insertions, or both. Guidance in determining which amino acid residues may be substituted, inserted, or deleted without abolishing biological activity may be found using computer programs well known in the art, for example, LASERGENE software (DNASTAR).

Alzheimer's disease (AD) is a progressive neurodegenerative disorder characterized by cognitive decline, irreversible memory loss, disorientation, and language impairment. AD affects 10% of the population aged greater than 65 and at least 50% of the population aged greater than 85 years. AD has been reported in patients as young as 40-50 years of age, but because the presence of the disease is difficult to detect without histopathological examination of brain tissue, the time of onset in living subjects is unknown. Several etiological factors have been implicated in the pathogenesis of Alzheimer's disease. These factors lead to the activation of a cascade process that brings about neuronal death and serious decline in cognitive function. These bed-ridden patients ultimately succumb to death due to inter-current infections related to aspiration, decubitus and stagnation of urine.

AD involves, biochemically, a pathological cleavage of amyloid precursor protein (APP). APP, in normal circumstances, is cleaved by α- and γ-secretases and takes part in axonal transport, synapse formation and synaptic repair in the CNS. The abnormal, sequential processing by the beta-site amyloid precursor protein-cleaving enzymes (BACE) and γ-secretase results in amyloid-β, which is highly neurotoxic. Molecules of amyloid-β, especially the amyloid-β₁₋₄₂ type, are prone to aggregation and accumulation in the cell membrane forming insoluble aggregates called “rafts”. Subsequently, these impair membrane conductivity, Ca²⁺ fluxes, control of the formation of reactive oxygen species (ROS), T-protein assembly, axonal transport, and the polarity of mitochondrial membrane. Ultimately, the pathologic cascade leads to Ca²⁺ toxicity, activation of apoptotic processes, inflammation and neuronal death. This vicious cycle can be initiated by a wide array of triggering mechanisms that can be traced back to genetic or environmental factors. Monogenic forms represent the infrequent, presenile or early-onset familial Alzheimer's disease (FAD), which is usually characterized by autosomal dominant point mutations of the genes of APP or the presenilin sub-domains of γ-secretase. Both types of mutations facilitate the accumulation of toxic amyloid-β₁₋₄₂, due to abnormal processing or breakdown.

Epidemiological studies show strong correlation between Alzheimer's disease and metabolic syndrome demonstrating that the lipoprotein profile (ApoE4 homozygous genotype), hyperinsulinemia and type II diabetes mellitus are among the most characteristic prognostic factors. The secretion of central nervous system (CNS) neurohormones, the master regulators of these endocrine and metabolic conditions, is considerably altered by senescence. Changes in corticotrophin releasing hormone (CRH), luteinizing hormone-releasing hormone (LHRH), and GHRH secretion related to obesity, hyperinsulinemia and altered leptin signaling may play a role in the development of Alzheimer's disease.

Parkinson's disease (PD) is a progressive neurodegenerative disease characterized by resting tremors, bradykinesia, muscular rigidity, and postural instability. PD typically develops after the age of 60, though 15% of diagnosed patients are under the age of 50. Family history of PD is an etiological factor for 5-10% of patients diagnosed with the disease, yet only 1% of cases have been shown to be clearly familial. It is estimated that 1.5 million Americans are currently living with PD.

Dementia with Lewy Bodies (DLB) is a progressive brain disease having symptoms that fluctuate between various degrees of manifestation. These symptoms include progressive dementia, Parkinsonian movement difficulties, hallucinations, and increased sensitivity to neuroleptic drugs. As with AD, advanced age is considered to be the greatest risk factor for DLB, with average onset usually between the ages of 50-85. Further, 20% of all dementia cases are caused by DLB and over 50% of PD patients develop “Parkinson's Disease Dementia” (PDD), a type of DLB. It is possible for DLB to occur alone, or in conjunction with other brain abnormalities, including those involved in AD and PD, as mentioned above.

The etiology of neurodegeneration can also involve a mixture of pathologies including a component of microvascular, or perfusion, deficits in the brain. For example, a disorder commonly referred to as “mixed dementia” often comprises both perfusion deficits and amyloid plaque pathology. The term “mixed dementia” possesses various meanings, but the term is commonly used to refer to the coexistence of AD and vascular dementia (VaD), in particular where the VaD is caused by numerous micro-thrombi in the vascular system of the brain. Though little is currently known about the true prevalence of mixed dementia, this form of neurodegeneration is clinically important because the combination of AD and VaD may have a greater impact on the brain than either condition independently. Symptoms are similar to those of AD or VaD or a combination of the two.

The occurrence of amyloid plaque deposits in the brain may be characteristic of numerous neurodegenerative diseases or other conditions including, but not limited to, Mediterranean fever, Muckle-Wells syndrome, idiopathic myeloma, amyloid polyneuropathy, amyloid cardiomyopathy, systemic neuritic amyloidosis, amyloid polyneuropathy, hereditary cerebral hemorrhage with amyloidosis, Down's syndrome, Scrapie, Creutzfeldt-Jacob disease, Kuru, Gerstamnn-Straussler-Scheinker syndrome, medullary carcinoma of the thyroid, isolated atrial amyloid, β2-microglobulin amyloid in dialysis patients, inclusion body myositis, β2-amyloid deposits in muscle wasting disease, type II diabetes, and combinations thereof.

Growth hormone releasing hormone (GHRH) is a peptide belonging to the secretin glucagon family of neuroendocrine and gastrointestinal hormones, a family that also includes vasoactive intestinal peptide (VIP), pituitary adenylate cyclase activating peptide (PACAP) and others. Human GHRH (hGHRH) peptide is comprised of 44 amino acid residues. The best known site of production of GHRH is the hypothalamus, but it was found that various peripheral organs also synthesize it. hGHRH is also produced, sometimes in large quantities, by human malignant tissues (cancers) of diverse origin.

GHRH exerts various physiological and pathophysiological functions. Hypothalamic GHRH is an endocrine releasing hormone that, acting through specific GHRH receptors on the pituitary, regulates the secretion of pituitary growth hormone (GH). The physiological functions of GHRH in extrapituitary tissues are less clear. However, there is increasing evidence for the role of GHRH as an autocrineparacrine growth factor in various cancers. Splice variant (SV) receptors for GHRH, different from those expressed in the pituitary, have been described in a wide range of human cancers and in some normal peripheral organs. The actions of tumoral autocrineparacrine GHRH could be exerted on these receptors. GHRH may also act independently of GH, by binding to their putative GHRH receptors on non-endocrine tissues. In addition, receptors for splice variants and other, as yet unidentified receptors of this family, could all be targets of local GHRH.

GHRH antagonists have been implicated in treating various disorders. GHRH antagonists inhibit the proliferation of malignancies by indirect endocrine mechanisms based on the inhibition of pituitary GH release and resulting in the decrease of serum levels of GH and IGF-1, as well as by direct effects on the tumor tissue. GHRH and its tumoral splice variant (SV) receptors are present in human cancers of the lung, prostate, breast, ovary, endometrium, stomach, intestine, pancreas, kidney, and bone. Tumoral GHRH has been shown

or it is suspected to act as an autocrine growth factor in these malignancies. Antagonistic analogs of GHRH can inhibit the stimulatory activity of GHRH and exert direct antiproliferative effects in vitro on cancer cells, and in vivo on tumors. In addition to the specific tumoral SV receptors for GHRH, receptors for VIP and other, as yet unidentified receptors of this family, are targets of GHRH antagonists.

Various modifications of GHRH peptides confer antagonistic properties. The GHRH fragment comprising residues 1 to 29, or GHRH(1-29), is the minimum sequence necessary for biological activity on the pituitary. This fragment retains 50% or more of the potency of native GHRH. Many synthetic analogs of GHRH, based on the structure of hGH-RH(1-29)NH₂ peptide have been prepared. hGHRH(1-29)NH₂ (SEQ ID NO: 3) has the following amino acid sequence:

Tyr-Ala-Asp-Ala-Ile⁵-Phe-Thr-Asn-Ser-Tyr¹⁰-Arg-Lys-Val-Leu-Gly¹⁵-Gln-Leu-Ser-Ala-Arg²⁰-Lys-Leu-Leu-Gln-Asp²⁵-Ile-Met-Ser-Arg²⁹-NH₂ (SEQ ID NO: 1).

A GHRH antagonist may comprise a GHRH peptide sequence to which amino acid deletions, insertions, and/or substitutions have been made. A GHRH antagonist may also be a fragment or modified fragment of GHRH having the capability to bind to the GHRH receptor and inhibiting the release of growth hormone. These antagonistic properties are believed to result from replacement of various amino acids and acylation with aromatic or nonpolar acids at the N-terminus of GHRH(1-29)NH₂.

Disclosed herein are a novel series of synthetic peptide analogs of hGHRH(1-29)NH₂. The novel synthetic peptides of this invention exhibit high antagonistic activities in blocking the release of pituitary growth hormone (GH) in animals, including humans. They also show extremely high binding capacity to the hGHRH receptor.

In some embodiments, the GHRH peptide antagonist has amino acid sequences represented by the formula I:

R¹-Tyr¹-D-Arg²-Asp³-A⁴-Ile⁵-A⁶-Thr⁷-A⁸-Har⁹-A¹⁰-A¹¹-A¹²-Val¹³-Leu¹⁴-A¹⁵-Gln¹⁶-A¹⁷-Ser¹⁸-Ala¹⁹-A²⁰-A²¹-Leu²²-Leu²³-Gln²⁴-Asp²⁵-Ile²⁶-Nle²⁷-D-Arg²⁸-A²⁹-R²-R³-NH₂,

wherein R¹ is PhAc, Nac, Oct, N-Me-Aib, Dca, Ac-Ada, Fer, Ac-Amc, Me-NH-Sub, PhAc-Ada, Ac-Ada-D-Phe, Ac-Ada-Phe, Dca-Ada, Nac, Nac-Ada, Ada-Ada, or CH₃(CH₂)₁₀-CO-Ada;

A⁴ is Ala or Me-Ala;

A⁶ is Cpa or Phe(F)₅;

A⁸ is Ala, Pal, Dip, or Me-Ala;

A¹⁰ is FPa5,Tyr(Alk) where Alk is Me or Et;

A¹¹ is His or Arg;

A¹² is Lys, Lys(0-11), Lys(Me)₂, or Orn;

A¹⁵ is Abu or Orn;

A¹⁷ is Leu or Glu;

A²⁰ is Har or His;

A²¹ is Lys, Lys(Me)₂ or Orn;

A²⁹ is Har, Arg or Agm;

R² is β-Ala, Amc, Apa, Ada, AE₂A, AE₄P, ε-Lys(α-NH₂), Agm, or absent; and

R³ is Lys(Oct), Ahx, or absent.

The above peptide formula may also be represented alternatively as:

[R¹-Tyr¹-D-Arg²-A⁴-A⁶-A⁸-Har⁹-A¹⁰-A¹¹-A¹²-A¹⁵-A¹⁷-A²⁰-A²¹-Nle²⁷-D-Arg²⁸-A²⁹-R²-R³]hGHRH(1-29)NH₂,
wherein R¹ PhAc, Nac, Oct, N-Me-Aib, Dca, Ac-Ada, Fer, Ac-Amc, Me-NH-Sub, PhAc-Ada, Ac-Ada-D-Phe, Ac-Ada-Phe, Dca-Ada, Nac-Ada, Ada-Ada, or CH₃(CH₂)₁₀-CO-Ada;

A⁴ is Ala or Me-Ala;

A⁶ is Cpa or Phe(F)₅;

A⁸ is Ala, Pal, Dip, or Me-Ala;

A¹⁰ is FPa5,Tyr(Alk) where Alk is Me or Et;

A¹¹ is His or Arg;

A¹² is Lys, Lys(0-1 l), Lys(Me)₂, or Orn;

A¹⁵ is Abu or Orn;

A¹⁷ is Leu or Glu;

A²⁰ is Har or His;

A²¹ is Lys, Lys(Me)₂ or Orn;

A²⁹ is Har, Arg or Agm;

R² is β-Ala, Amc, Apa, Ada, AE₂A, AE₄P, ε-Lys(α-NH₂), Agm, or absent; and

R³ is Lys(Oct), Ahx, or absent.

In some embodiments, the peptides may lack R² and/or R³ C-terminal modifications. In some embodiments, when R¹ is PhAc, Nac, or Oct, R² is not absent.

In some embodiments, R¹ may be PhAc, Nac, Oct, N-Me-Aib, Dca, Ac-Ada, Fer, Ac-Amc, Me-NH-Sub, or PhAc-Ada. In some embodiments, R¹ may be PhAc-Ada or PhAc. In some embodiments, A⁸ may be Ala or Me-Ala. In some embodiments, A¹⁰ may be Tyr(Me) or FPa5. In some embodiments, A¹⁷ may be Leu or Glu. In some embodiments, R² may be Ada or Agm.

In some embodiments, the GHRH peptide antagonist has amino acid sequences of formula II:

R¹-Tyr¹-D-Arg²-Asp³-Ala⁴-Ile⁵-A⁶-Thr⁷-A⁸-Har⁹-A¹⁰-His¹¹-Orn¹²-Val¹³-Leu¹⁴-Abu¹⁵-Gln¹⁶-A¹⁷-Ser¹⁸-Ala¹⁹-His²⁰-Orn²¹-Leu²²-Leu²³-Gln²⁴-Asp²⁵-Ile²⁶-Nle²⁷-D-Arg²⁸-Har²⁹-R²-NH₂,

wherein R¹ is PhAc-Ada or PhAc;

A⁶ is Phe(F)₅ or Cpa;

A⁸ is Ala or Me-Ala;

A¹⁰ is Tyr(Me) or FPa5;

A¹⁷ is Leu or Glu; and

R² is Ada, Agm, or absent.

The nomenclature used to define the amino acid residues and synthetic peptides is according to the IUPAC-IUB Commission on Biochemical Nomenclature (European J. Biochem., 1984, 138, 9-37). The naturally occurring amino acids found in proteins are depicted by the following three letter codes: Gly, Ala, Val, Leu, Ile, Ser, Thr, Lys, Arg, Asp, Asn, Glu, Gln, Cys, Met Phe, Tyr, Pro, Trp and His.

Other abbreviations used are:
Abu alpha-aminobutyric acid
Ac acetyl
AcOH acetic acid
Ac₂O acetic anhydride
Ada 12-aminododecanoyl
AE₂A 8-amino-3,6-dioxaoctanoyl
AE₄P 15-amino-4,7,10,13-tetraoxapentadecanoyl
Agm agmatine
Ahx 6-aminohexanoyl
Amc 8-aminocaprylyl
Apa 5-aminopentanoyl
Aib alpha-aminoisobutyroyl
All allyl
Alloc allyloxycarbonyl
Amp para-amidino-phenylalanine
Bpa para-benzoyl-phenylalanine
Boc tert-butyloxycarbonyl
Bom benzyloxymethyl
2BrZ 2-bromo-benzyloxycarbonyl
Bzl benzyl
Cha cyclohexylalanine
Chg cyclohexylglycine
cHx cyclohexyl
Cit citrulline (2-amino-5-ureidovaleroyl
2CIZ 2-chloro-benzyloxycarbonyl
Cpa para-chlorophenylalanine
Dat des-amino-tyrosine
Dca dichloroacetyl
DCM dichloromethane
DIG N,N′-diisopropylcarbodiimide
DIEA diisopropylethylamine
Dip (3,3-diphenyl)alanine
DMF dimethylformamide
Et ethyl
Fer ferulyl
FGF fibroblast growth factor
Fm fluorenylmethyl
Fmoc fluorenylmethoxycarbonyl
For formyl
GH growth hormone
GHRH GH releasing hormone
Gup para-guanidine-phenylalanine
Har homoarginine
HBTU 2-(1H-Benzotriazol-1-yl)-1,1,3,3-tetramethyluronium hexaflourophosphate
Hca hydrocinnamoyl
Hca-OH hydrocinnamic acid
hGHRH human GH-RH
HOBt 1-hydroxybenzotriazole
HPLC high performance liquid chromatography
Ibu isobutyryl
IndAc indole-3-acetyl
Ipa indole-3-propionyl
Lys(0-11) Lys(A0-A1-A2-A3-A4-A5-A6-A7-A8-A9 A10-A11-)
ε-Lys(α-NH₂) a Lys residue, the 8-amino group of which is acylated by the carbonyl group of an N-terminally located amino acid; the a-amino group of the Lys residue is free
MBHA para-methylbenzhydrylamine
Me methyl
MeOH methanol
MeCN acetonitrile
Nac naphthylacetyl
Nal naphthylalanine
Nle norleucine
NMM N-methylmorpholine
Npr naphthylpropionyl
Oct octanoyl
Orn ornithine
Peg pegyl
Pal pyridylalanine
PAM phenylacetamidomethyl
Ph phenyl
PhAc phenylacetyl
PhAc-OH phenylacetic acid
Phe(pCI) para-chloro-phenylalanine
Phe(pNH₂) para-amino-phenylalanine
Phe(pNO₂) para-nitro-phenylalanine
rGHRH rat GHRH
RP-HPLC reversed phase HPLC
Sub suberyl
SPA para-sulfonyl-phenoxyacetyl
TFA trifluoroacetic acid
Tos para-toluenesulfonyl
Tpi 1,2,3,4-tetrahydronorharman-3-carboxylic acid
Tyr(Me) O-methyl-tyrosine
Tyr(Et) O-ethyl-tyrosine
Z benzyloxycarbonyl

The amino acid sequences of the synthetic peptides are numbered in correspondence with the amino acid residues in hGHRH(1-29) (SEQ ID NO: 1). Thus, for example, the Ala⁴ and A⁸ in the synthetic peptides occupy the same position in the sequence as the Ala⁴ and A⁸ residues in hGHRH(1-29). The convention under which the N-terminal of a peptide is placed to the left, and the C-terminal to the right is also followed herein. In some embodiments, the peptides may have N-terminal modifications, represented by R¹. In some embodiments, the peptides may have C-terminal modifications, represented by R² and R³. In some embodiments, the peptides may lack R² and/or R³ C-terminal modifications.

In some embodiments, specific GHRH peptide antagonists include:
Phac-Ada-Tyr¹-D-Arg²-Asp³-Ala⁴-Ile⁵-Phe(F)₅⁶-Thr⁷-Ala⁸-Har⁹-Tyr(Me)¹⁰-His¹¹-Orn¹²-Val¹³-Leu¹⁴-Abu¹⁵-Gln¹⁶-Leu¹⁷-Ser¹⁸-Ala¹⁹-His²⁰-Orn²¹-Leu²²-Leu²³-Gln²⁴-Asp²⁵-Ile²⁶-Nle²⁷-D-Arg²⁸-Har²⁹-NH₂ (MIA-602).
Phac-Ada-Tyr¹-D-Arg²-Asp³-Ala⁴-Ile⁵-Phe(F)₅⁶-Thr⁷-Ala⁸-Har⁹-Tyr(Me)¹⁰-His¹¹-Orn¹²-Val¹³-Leu¹⁴-Abu¹⁵-Gln¹⁶-Leu¹⁷-Ser¹⁸-Ala¹⁹-His²⁰-Orn²¹-Leu²²-Leu²³-Gln²⁴-Asp²⁵-Ile²⁶-Nle²⁷-D-Arg²⁸-Har²⁹-Agm-NH₂ (MIA-604).
Phac-Ada-Tyr¹-D-Arg²-Asp³-Ala⁴-Ile⁵-Phe(F)₅⁶-Thr⁷-Me-Ala⁸-Har⁹-Tyr(Me)¹⁰-His¹¹-Orn¹²-Val¹³-Leu¹⁴-Abu¹⁵-Gln¹⁶-Leu¹⁷-Ser¹⁸-Ala¹⁹-His²⁰-Orn²¹-Leu²²-Leu²³-Gln²⁴-Asp²⁵-Ile²⁶-Nle²⁷-D-Arg²⁸-Har²⁹-Agm-NH₂ (MIA-606).
Phac-Tyr¹-D-Arg²-Asp³-Ala⁴-Ile⁵-Cpa⁶-Thr⁷-Ala⁸-Har⁹-Fpa5¹⁰-His¹¹-Orn¹²-Val¹³-Leu¹⁴-Abu¹⁵-Gln¹⁶-Leu¹⁷-Ser¹⁸-Ala¹⁹-His²⁰-Orn²¹-Leu²²-Leu²³-Gln²⁴-Asp²⁵-Ile²⁶-Nle²⁷-D-Arg²⁸-Har²⁹-Ada-NH₂ (MIA-610).
Phac-Ada-Tyr¹-D-Arg²-Asp³-Ala⁴-Ile⁵-Cpa⁶-Thr⁷-Ala⁸-Har⁹-Fpa5¹⁰-His¹¹-Orn¹²-Val¹³-Leu¹⁴-Abu¹⁵-Gln¹⁶-Glu¹⁷-Ser¹⁸-Ala¹⁹-His²⁰-Orn²¹-Leu²²-Leu²³-Gln²⁴-Asp²⁵-Ile²⁶-Nle²⁷-D-Arg²⁸-Har²⁹-Ada-NH₂ (MIA-640).
Phac-Ada-Tyr¹-D-Arg²-Asp³-Ala⁴-Ile⁵-Cpa⁶-Thr⁷-Ala⁸-Har⁹-Fpa5¹⁰-His¹¹-Orn¹²-Val¹³-Leu¹⁴-Abu¹⁵-Gln¹⁶-Leu¹⁷-Ser¹⁸-Ala¹⁹-His²⁰-Orn²¹-Leu²²-Leu²³-Gln²⁴-Asp²⁵-Ile²⁶-Nle²⁷-D-Arg²⁸-Har²⁹-NH₂ (MIA-690).

Suitable synthetic GHRH peptide antagonists are disclosed in Table 1. The peptides disclosed herein are various modifications of the core GHRH peptide antagonist represented by formula I. For example, P-1109 has N-terminal modification PhAc; Tyr at amino acid position 1; D-Arg at position 2; Ala at position 4, Cpa at position 6, and so on, and the intervening amino acids (i.e., those that are not specifically identified in the table) at positions 3, 4, 5, 7, 12, 13, 14, 16, 18, 19, and 21-26 are the same amino acids of formula I.

TABLE 1

| | |
|---|---|
| P-1109 | [PhAc-Tyr¹, D-Arg², Ala⁴, Cpa⁶, Ala⁸, Har⁹, Tyr(Me)¹⁰, His¹¹, |
| | Abu¹⁵, Glu¹⁷, His²⁰, Nle²⁷, D-Arg²⁸, Har²⁹]hGH-RH(1-29)NH₂ |
| P-1111 | [PhAc-Tyr¹, D-Arg², (Me-Ala)⁴, Cpa⁶, Ala⁸, Har⁹, Tyr(Me)¹⁰, His¹¹, Abu¹⁵, |
| | His²⁰, Nle²⁷, D-Arg²⁸, Har²⁹]hGH-RH(1-29)NH₂ |
| P-1113 | [PhAc-Tyr¹, D-Arg², Ala⁴, Cpa⁶, (Me-Ala)⁸, Har⁹, Tyr(Me)¹⁰, |
| | His¹¹, Abu¹⁵, His²⁰, Nle²⁷, D-Arg²⁸, Har²⁹]hGH-RH(1-29)NH₂ |
| P-1115 | [PhAc-Tyr¹, D-Arg², Ala⁴, Cpa⁶, Ala⁸, Har⁹, Tyr(Me)¹⁰, His¹¹, |
| | Abu¹⁵, His²⁰, (Lys(Me)₂)²¹, Nle²⁷, D-Arg²⁸, Har²⁹]hGH-RH(1-29)NH₂ |
| P-1117 | [PhAc-Tyr¹, D-Arg², Ala⁴, Cpa⁶, Ala⁸, Har⁹, Tyr(Me)¹⁰, His¹¹, |
| | (Lys(Me)₂)¹², Abu¹⁵, His²⁰, (Lys(Me)₂)²¹, Nle²⁷, D-Arg²⁸, |
| | Har²⁹]hGH-RH(1-29)NH₂ |
| P-11107 | [(N-Me-Aib)-Tyr¹, D-Arg², Ala⁴, Cpa⁶, Ala⁸, Har⁹, Tyr(Me)¹⁰, |
| | His¹¹, Abu¹⁵, His²⁰, Nle²⁷, D-Arg²⁸, Har²⁹]hGH-RH(1-29)NH₂ |
| P-11109 | [Dca-Tyr¹, D-Arg², Ala⁴, Cpa⁶, Ala⁸, Har⁹, Tyr(Me)¹⁰, His¹¹, |
| | Abu¹⁵, His²⁰, Nle²⁷, D-Arg²⁸, Har²⁹]hGH-RH(1-29)NH₂ |
| P-11111 | [(Ac-Ada)-Tyr¹, D-Arg², Ala⁴, Cpa⁶, Ala⁸, Har⁹, Tyr(Me)¹⁰, |
| | His¹¹, Abu¹⁵, His²⁰, Nle²⁷, D-Arg²⁸, Har²⁹]hGH-RH(1-29)NH₂ |
| P-11113 | [Fer-Tyr¹, D-Arg², Ala⁴, Cpa⁶, Ala⁸, Har⁹, Tyr(Me)¹⁰, His¹¹, |
| | Abu¹⁵, His²⁰, Nle²⁷, D-Arg²⁸, Har²⁹]hGH-RH(1-29)NH₂ |
| P-11115 | [(Ac-Amc)-Tyr¹, D-Arg², Ala⁴, Cpa⁶, Ala⁸, Har⁹, Tyr(Me)¹⁰, |
| | His¹¹, Abu¹⁵, His²⁰, Nle²⁷, D-Arg²⁸, Har²⁹]hGH-RH(1-29)NH₂ |
| P-11117 | [(PhAc-Ada)⁰-Tyr¹, D-Arg², Ala⁴, Cpa⁶, Ala⁸, Har⁹, |
| | Tyr(Me)¹⁰, His¹¹, Abu¹⁵, His²⁰, Nle²⁷, D-Arg²⁸, Har²⁹]hGH- |
| | RH(1-29)NH₂ |
| P-11119 | [(Ac-Ada-D-Phe)-Tyr¹, D-Arg², Ala⁴, Cpa⁶, Ala⁸, Har⁹, |
| | Tyr(Me)¹⁰, His¹¹, Abu¹⁵, His²⁰, Nle²⁷, D-Arg²⁸, Har²⁹]hGH- |
| | RH(1-29)NH₂ |
| P-11121 | [(Ac-Ada-Phe)-Tyr¹, D-Arg², Ala⁴, Cpa⁶, Ala⁸, Har⁹, |
| | Tyr(Me)¹⁰, His¹¹, Abu¹⁵, His²⁰, Nle²⁷, D-Arg²⁸, Har²⁹]hGH- |
| | RH(1-29)NH₂ |
| P-11123 | [(Dca-Ada)-Tyr¹, D-Arg², Ala⁴, Cpa⁶, Ala⁸, Har⁹, Tyr(Me)¹⁰, |
| | His¹¹, Abu¹⁵, His²⁰, Nle²⁷, D-Arg²⁸, Har²⁹]hGH-RH(1-29)NH₂ |
| P-11125 | [(CH₃—(CH₂)₁₀—CO-Ada)-Tyr¹, D-Arg², Ala⁴, Cpa⁶, Ala⁸, Har⁹, |
| | Tyr(Me)¹⁰, His¹¹, Abu¹⁵, His²⁰, Nle²⁷, D-Arg²⁸, Har²⁹]hGH- |
| | RH(1-29)NH₂ |
| P-11207 | [PhAc-Tyr¹, D-Arg², Ala⁴, Cpa⁶, Ala⁸, Har⁹, Tyr(Me)¹⁰, His¹¹, |
| | Abu¹⁵, His²⁰, Nle²⁷, D-Arg²⁸, Har²⁹-Amc]hGH-RH(1-29)NH₂ |
| P-11209 | [PhAc-Tyr¹, D-Arg², Ala⁴, Cpa⁶, Ala⁸, Har⁹, Tyr(Me)¹⁰, His¹¹, |
| | Abu¹⁵, His²⁰, Nle²⁷, D-Arg²⁸, Har²⁹-Apa]hGH-RH(1-29)NH₂ |
| P-11211 | [PhAc-Tyr¹, D-Arg², Ala⁴, Cpa⁶, Ala⁸, Har⁹, Tyr(Me)¹⁰, His¹¹, |
| | Abu¹⁵, His²⁰, Nle²⁷, D-Arg²⁸, Har²⁹-Ada]hGH-RH(1-29)NH₂ |
| P-11213 | [Oct-Tyr¹, D-Arg², Ala⁴, Cpa⁶, Ala⁸, His⁹, Tyr(Et)¹⁰, His¹¹, |
| | Orn¹², Abu¹⁵, His²⁰, Orn²¹, Nle²⁷, D-Arg²⁸, Har²⁹-Ada]hGH- |
| | RH(1-29)NH₂ |
| P-11215 | [PhAc-Tyr¹, D-Arg², Ala⁴, Cpa⁶, Ala⁸, Har⁹, Tyr(Me)¹⁰, His¹¹, |
| | Abu¹⁵, His²⁰, Nle²⁷, D-Arg²⁸, Arg²⁹-Ada]hGH-RH(1-29)NH₂ |
| P-11307 | [(Ac-Amc)-Tyr¹, D-Arg², Ala⁴, Cpa⁶, Ala⁸, Har⁹, Tyr(Me)¹⁰, |
| | His¹¹, Orn¹², Abu¹⁵, His²⁰, Nle²⁷, D-Arg²⁸, Har²⁹-Amc]hGH- |
| | RH(1-29)NH₂ |
| P-11309 | [(Me-NH-Sub)-Tyr¹, D-Arg², Ala⁴, Cpa⁶, Ala⁸, Har⁹, |
| | Tyr(Me)¹⁰, His¹¹, Abu¹⁵, His²⁰, Nle²⁷, D-Arg²⁸, Har²⁹- |
| | Amc]hGH-RH(1-29)NH₂ |
| P-11311 | [(Ac-Amc)-Tyr¹, D-Arg², Ala⁴, Cpa⁶, Ala⁸, Har⁹, Tyr(Me)¹⁰, |
| | His¹¹, Orn¹², Abu¹⁵, His²⁰, Nle²⁷, D-Arg²⁸, Agm²⁹]hGH-RH(1-29) |
| P-11313 | [(Ac-Amc)-Tyr¹, D-Arg², Ala⁴, Cpa⁶, Ala⁸, Har⁹, Tyr(Me)¹⁰, |
| | His¹¹, Orn¹², Abu¹⁵, His²⁰, Nle²⁷, D-Arg²⁸, Har²⁹-Agm]hGH- |
| | RH(1-29) |
| P-11315 | [(Ac-Amc)-Tyr¹, D-Arg², Ala⁴, Cpa⁶, Ala⁸, Har⁹, Tyr(Me)¹⁰, |
| | His¹¹, Abu¹⁵, His²⁰, Nle²⁷, D-Arg²⁸, Har²⁹-Ada]hGH-RH(1-29)NH₂ |
| P-11317 | [(Ac-Ada)-Tyr¹, D-Arg², Ala⁴, Cpa⁶, Ala⁸, Har⁹, Tyr(Me)¹⁰, |
| | His¹¹, Abu¹⁵, His²⁰, Nle²⁷, D-Arg²⁸, Har²⁹-Ada]hGH-RH(1-29)NH₂ |
| P-11319 | [(Ac-Ada)-Tyr¹, D-Arg², Ala⁴, Cpa⁶, Ala⁸, His⁹, Tyr(Et)¹⁰, |
| | His¹¹, Orn¹², Abu¹⁵, His²⁰, Orn²¹, Nle²⁷, D-Arg²⁸, Har²⁹- |
| | Ada]hGH-RH(1-29)NH₂ |
| P-11321 | [(Ac-Ada)-Tyr¹, D-Arg², Ala⁴, Cpa⁶, Ala⁸, Har⁹, Tyr(Me)¹⁰, |
| | His¹¹, Abu¹⁵, His²⁰, Nle²⁷, D-Arg²⁸, Arg²⁹-Ada]hGH-RH(1-29)NH₂ |
| P-11407 | [(Ac-Amc)-Tyr¹, D-Arg², (Me-Ala)⁴, Cpa⁶, Ala⁸, Har⁹, |
| | Tyr(Me)¹⁰, His¹¹, Abu¹⁵, His²⁰, Nle²⁷, D-Arg²⁸, Har²⁹]hGH- |
| | RH(1-29)NH₂ |
| P-11408 | [(Ac-Amc)-Tyr¹, D-Arg², Ala⁴, Cpa⁶, (Me-Ala)⁸, Har⁹, |
| | Tyr(Me)¹⁰, His¹¹, Abu¹⁵, His²⁰, Nle²⁷, D-Arg²⁸, Har²⁹]hGH- |
| | RH(1-29)NH₂ |
| P-11409 | [(Ac-Amc)-Tyr¹, D-Arg², (Me-Ala)⁴, Cpa⁶, (Me-Ala)⁸, Har⁹, |
| | Tyr(Me)¹⁰, His¹¹, Abu¹⁵, His²⁰, Nle²⁷, D-Arg²⁸, Har²⁹]hGH- |
| | RH(1-29)NH₂ |
| P-11411 | [PhAc-Tyr¹, D-Arg², Ala⁴, Cpa⁶, (Me-Ala)⁸, Har⁹, Tyr(Me)¹⁰, |
| | His¹¹, Abu¹⁵, His²⁰, Nle²⁷, D-Arg²⁸, Har²⁹-Amc]hGH-RH(1-29)NH₂ |
| P-11413 | [PhAc-Tyr¹, D-Arg², Ala⁴, Cpa⁶, 3-Pal⁸, Har⁹, Tyr(Me)¹⁰, |
| | His¹¹, Abu¹⁵, His²⁰, Nle²⁷, D-Arg²⁸, Har²⁹-Amc]hGH-RH(1-29)NH₂ |
| P-11415 | [(Ac-Amc)-Tyr¹, D-Arg², Ala⁴, Cpa⁶, (Me-Ala)⁸, Har⁹, |
| | Tyr(Me)¹⁰, His¹¹, Abu¹⁵, His²⁰, Nle²⁷, D-Arg²⁸, Har²⁹- |
| | Agm]hGH-RH(1-29) |
| P-11417 | [(Ac-Amc)-Tyr¹, D-Arg², Ala⁴, Cpa⁶, (Me-Ala)⁸, Har⁹, |
| | Tyr(Me)¹⁰, His¹¹, Abu¹⁵, His²⁰, Nle²⁷, D-Arg²⁸, Har²⁹- |
| | Amc]hGH-RH(1-29)NH₂ |
| P-11419 | [PhAc-Tyr¹, D-Arg², Ala⁴, Cpa⁶, Ala⁸, Har⁹, Tyr(Me)¹⁰, His¹¹, |
| | Orn¹², Abu¹⁵, His²⁰, Orn²¹, Nle²⁷, D-Arg²⁸, Har²⁹-AE₂A]hGH- |
| | RH(1-29)NH₂ |
| P-11421 | [(N-Me-Aib)-Tyr¹, D-Arg², Ala⁴, Cpa⁶, Ala⁸, Har⁹, Tyr(Me)¹⁰, |
| | His¹¹, Orn¹², Abu¹⁵, His²⁰, Orn²¹, Nle²⁷, D-Arg²⁸, Har²⁹- |
| | AE₂A]hGH-RH(1-29)NH₂ |
| P-11423 | [PhAc-Tyr¹, D-Arg², Ala⁴, Cpa⁶, Dip⁸, Har⁹, Tyr(Me)¹⁰, His¹¹, |
| | Orn¹², Abu¹⁵, His²⁰, Orn²¹, Nle²⁷, D-Arg²⁸, Har²⁹-AE₂A]hGH- |
| | RH(1-29)NH₂ |
| P-11425 | [PhAc-Tyr¹, D-Arg², Ala⁴, Cpa⁶, Ala⁸, Har⁹, Tyr(Me)¹⁰, His¹¹, |
| | Lys(0-11)¹², Abu¹⁵, His²⁰, Orn²¹, Nle²⁷, D-Arg²⁸, Har²⁹]hGH- |
| | RH(1-29)NH₂, where (0-11) denotes the following peptide |
| | sequence: PhAc-Tyr-D-Arg-Asp-Ala-Ile-Cpa-Thr-Ala-Har- |
| | Tyr(Me)-His-; and the C-terminal carbonyl group of the (0-11) |
| | peptide sequence forms an amide bond with the epsilon |
| | amino group of Lys¹² |
| P-11427 | [(N-Me-Aib)-Tyr¹, D-Arg², Ala⁴, Cpa⁶, Ala⁸, Har⁹, Tyr(Me)¹⁰, |
| | His¹¹, Lys(0-11)¹², Abu¹⁵, His²⁰, Orn²¹, Me²⁷, D-Arg²⁸, |
| | Har²⁹]hGH-RH(1-29)NH₂; where (0-11) denotes the following |
| | peptide sequence: (N-Me-Aib)-Tyr-D-Arg-Asp-Ala-Ile-Cpa- |
| | Thr-Ala-Har-Tyr(Me)-His-; and the C-terminal carbonyl |
| | group of the (0-11) peptide sequence forms an amide bond |
| | with the epsilon amino group of Lys¹² |
| P-11429 | [PhAc-Tyr¹, D-Arg², Ala⁴, Cpa⁶, Ala⁸, Har⁹, Tyr(Me)¹⁰, His¹¹, |
| | Orn¹², Abu¹⁵, His²⁰, Orn²¹, Nle²⁷, D-Arg²⁸, Har²⁹-β-Ala- |
| | Lys(Oct)] hGH-RH(1-29)NH₂ |
| P-11431 | [(N-Me-Aib)-Tyr¹, D-Arg², Ala⁴, Cpa⁶, Ala⁸, Har⁹, Tyr(Me)¹⁰, |
| | His¹¹, Orn¹², Abu¹⁵, His²⁰, Orn²¹, Nle²⁷, D-Arg²⁸, Har²⁹-β-Ala- |
| | Lys(Oct)] hGH-RH(1-29)NH₂ |
| P-11433 | [Nac-Tyr¹, D-Arg², Ala⁴, Cpa⁶, Ala⁸, Har⁹, Tyr(Me)¹⁰, His¹¹, |
| | Orn¹², Abu¹⁵, His²⁰, Orn²¹, Nle²⁷, D-Arg²⁸, Har²⁹-AE₂A] hGH- |
| | RH(1-29)NH₂ |
| P-11435 | [(Ac-Ada)-Tyr¹, D-Arg², Ala⁴, Cpa⁶, Ala⁸, Har⁹, Tyr(Me)¹⁰, |
| | His¹¹, Orn¹², Abu¹⁵, His²⁰, Orn²¹, Nle²⁷, D-Arg²⁸, Har²⁹]hGH- |
| | RH(1-29)NH₂ |
| P-11437 | [(Dca-Ada)-Tyr¹, D-Arg², Ala⁴, Cpa⁶, Ala⁸, Har⁹, Tyr(Me)¹⁰, |
| | His¹¹, Orn¹², Abu¹⁵, His²⁰, Orn²¹, Nle²⁷, D-Arg²⁸, Har²⁹]hGH- |
| | RH(1-29)NH₂ |
| P-11439 | [(Ac-Ada)-Tyr¹, D-Arg², Ala⁴, Cpa⁶, Ala⁸, Har⁹, Tyr(Me)¹⁰, |
| | His¹¹, Abu¹⁵, His²⁰, (Lys(Me)₂)²¹, Nle²⁷, D-Arg²⁸, Har²⁹]hGH- |
| | RH(1-29)NH₂ |
| P-11441 | [(Ac-Ada)-Tyr¹, D-Arg², Ala⁴, Cpa⁶, Ala⁸, Har⁹, Tyr(Me)¹⁰, |
| | His¹¹, (Lys(Me)₂)¹², Abu¹⁵, His²⁰, (Lys(Me)₂)²¹, Nle²⁷, D- |
| | Arg²⁸, Har²⁹]hGH-RH(1-29)NH₂ |
| P-11443 | [(Dca-Ada)-Tyr¹, D-Arg², Ala⁴, Cpa⁶, Ala⁸, Har⁹, Tyr(Me)¹⁰, |
| | His¹¹, Abu¹⁵, His²⁰, (Lys(Me)₂)²¹, Nle²⁷, D-Arg²⁸, Har²⁹]hGH- |
| | RH(1-29)NH₂ |
| P-11445 | [(Dca-Ada)-Tyr¹, D-Arg², Ala⁴, Cpa⁶, Ala⁸, Har⁹, Tyr(Me)¹⁰, |
| | His¹¹, (Lys(Me)₂)¹², Abu¹⁵, His²⁰, (Lys(Me)₂)²¹, Nle²⁷, D- |
| | Arg²⁸, Har²⁹]hGH-RH(1-29)NH₂ |
| P-11447 | [PhAc-Tyr¹, D-Arg², Ala⁴, Cpa⁶, Ala⁸, Har⁹, Tyr(Me)¹⁰, His¹¹, |
| | Orn¹², Abu¹⁵, Har²⁰, Orn²¹, Nle²⁷, D-Arg²⁸, Har²⁹]hGH-RH(1-29) |
| | NH₂ |
| P-11449 | [(Ac-Ada)-Tyr¹, D-Arg², Ala⁴, Cpa⁶, Ala⁸, Har⁹, Tyr(Me)¹⁰, |
| | His¹¹, Orn¹², Abu¹⁵, Har²⁰, Orn²¹, Nle²⁷, D-Arg²⁸, Har²⁹]hGH- |
| | RH(1-29)NH₂ |
| P-11451 | [(Nac-Ada)-Tyr¹, D-Arg², Ala⁴, Cpa⁶, Ala⁸, Har⁹, Tyr(Me)¹⁰, |
| | His¹¹, Orn¹², Abu¹⁵, His²⁰, Orn²¹, Nle²⁷, D-Arg²⁸, Har²⁹- |
| | AE₂A]hGH-RH(1-29)NH₂ |
| P-11453 | [(Dca-Ada)-Tyr¹, D-Arg², Ala⁴, Cpa⁶, Ala⁸, Har⁹, Tyr(Me)¹⁰, |
| | His¹¹, Orn¹², Abu¹⁵, His²⁰, Orn²¹, Nle²⁷, D-Arg²⁸, Har²⁹- |
| | AE₂A]hGH-RH(1-29)NH₂ |
| P-11455 | [(Ac-Ada)-Tyr¹, D-Arg², Ala⁴, Cpa⁶, Ala⁸, Har⁹, Tyr(Me)¹⁰, |
| | His¹¹, Orn¹², Abu¹⁵, His²⁰, Orn²¹, Nle²⁷, D-Arg²⁸, Har²⁹- |
| | AE₂A]hGH-RH(1-29)NH₂ |
| P-11457 | [PhAc-Tyr¹, D-Arg², Ala⁴, Cpa⁶, Ala⁸, Har⁹, Tyr(Me)¹⁰, His¹¹, |
| | Orn¹², Abu¹⁵, His²⁰, Orn²¹, Nle²⁷, D-Arg²⁸, Har²⁹-Ada]hGH- |
| | RH(1-29)NH₂ |
| P-11459 | [(PhAc-Ada)-Tyr¹, D-Arg², Ala⁴, Cpa⁶, Ala⁸, Har⁹, |
| | Tyr(Me)¹⁰, His¹¹, Orn¹², Abu¹⁵, His²⁰, Orn²¹, Nle²⁷, D-Arg²⁸, |
| | Har²⁹]hGH-RH(1-29)NH₂ |
| P-11461 | [(Ac-Ada-Phe)-Tyr¹, D-Arg², Ala⁴, Cpa⁶, Ala⁸, Har⁹, |
| | Tyr(Me)¹⁰, His¹¹, Orn¹², Abu¹⁵, His²⁰, Orn²¹, Nle²⁷, D-Arg²⁸, |
| | Har²⁹]hGH-RH(1-29)NH₂ |
| P-11463 | [PhAc-Tyr¹, D-Arg², Ala⁴, Cpa⁶, Ala⁸, Har⁹, Tyr(Me)¹⁰, His¹¹, |
| | Orn¹², Abu¹⁵, His²⁰, Nle²⁷, D-Arg²⁸, Har²⁹-Amc]hGH-RH(1-29)NH₂ |
| P-11465 | [PhAc-Tyr¹, D-Arg², Ala⁴, Cpa⁶, Ala⁸, Har⁹, Tyr(Me)¹⁰, His¹¹, |
| | Abu¹⁵, His²⁰, Orn²¹, Nle²⁷, D-Arg²⁸, Har²⁹-Ada]hGH-RH(1-29)NH₂ |
| P-11467 | [(Ada-Ada)-Tyr¹, D-Arg², Ala⁴, Cpa⁶, Ala⁸, Har⁹, Tyr(Me)¹⁰, |
| | His¹¹, Orn¹², Abu¹⁵, His²⁰, Orn²¹, Nle²⁷, D-Arg²⁸, Har²⁹- |
| | Ada]hGH-RH(1-29)NH₂ |
| P-11469 | [(Ac-Ada)-Tyr¹, D-Arg², Ala⁴, Cpa⁶, Ala⁸, Har⁹, Tyr(Me)¹⁰, |
| | His¹¹, Orn¹², Abu¹⁵, His²⁰, Orn²¹, Nle²⁷, D-Arg²⁸, Har²⁹- |
| | Ada]hGH-RH(1-29)NH₂ |
| P-11471 | [(Ac-Ada)-Tyr¹, D-Arg², Ala⁴, Cpa⁶, Ala⁸, Har⁹, Tyr(Me)¹⁰, |
| | His¹¹, Lys(0-11)¹², Abu¹⁵, His²⁰, Orn²¹, Nle²⁷, D-Arg²⁸, |
| | Har²⁹]hGH-RH(1-29)NH₂ where (0-11) denotes the following |
| | peptide sequence: PhAc-Tyr-D-Arg-Asp-Ala-Ile-Cpa-Thr- |
| | Ala-Har- |
| | Tyr(Me)-His-; and the C-terminal carbonyl group of the (0-11) |
| | peptide sequence forms an amide bond with the epsilon |
| | amino group of Lys¹² |
| P-11473 | [(PhAc-Ada)-Tyr¹, D-Arg², Ala⁴, Cpa⁶, Ala⁸, Har⁹, |
| | Tyr(Me)¹⁰, His¹¹, Orn¹², Abu¹⁵, His²⁰, Orn²¹, Nle²⁷, D-Arg²⁸, |
| | Har²⁹-Ada]hGH-RH(1-29)NH₂ |
| P-11475 | [(Ac-Ada-D-Phe)-Tyr¹, D-Arg², Ala⁴, Cpa⁶, Ala⁸, Har⁹, |
| | Tyr(Me)¹⁰, His¹¹, Orn¹², Abu¹⁵, His²⁰, Orn²¹, Nle²⁷, D-Arg²⁸, |
| | Har²⁹-Ada³⁰]hGH-RH(1-30)NH₂ |
| P-11477 | [(Ac-Ada)-Tyr¹, D-Arg², Ala⁴, Cpa⁶, Ala⁸, Har⁹, Tyr(Me)¹⁰, |
| | His¹¹, Orn¹², Abu¹³, Glu¹⁷, His²⁰, Nle²⁷, D-Arg²⁸, Har²⁹]hGH- |
| | RH(1-29)NH₂ |
| P-11479 | [(Ac-Ada)-Tyr¹, D-Arg², Ala⁴, (Phe(F)₅)⁶, Ala⁸, Har⁹, |
| | Tyr(Me)¹⁰, His¹¹, Orn¹², Abu¹⁵, His²⁰, Orn²¹, Nle²⁷, D-Arg²⁸, |
| | Har²⁹]hGH-RH(1-29)NH₂ |
| P-11481 | [(Ac-Ada)-Tyr¹, D-Arg², Ala⁴, Cpa⁶, Ala⁸, Har⁹, Tyr(Me)¹⁰, |
| | His¹¹, Orn¹², Abu¹⁵, His²⁰, Orn²¹, Nle²⁷, D-Arg²⁸, Har²⁹-ε- |
| | Lys(α-NH₂)-Ahx]hGH-RH(1-29)NH₂ |
| P-11483 | [(Ac-Ada)-Tyr¹, D-Arg², Ala⁴, Cpa⁶, Ala⁸, Har⁹, Tyr(Me)¹⁰, |
| | His¹¹, Orn¹², Abu¹⁵, His²⁰, Orn²¹, Nle²⁷, D-Arg²⁸, Har²⁹- |
| | AE₄P]hGH-RH(1-29)NH₂ |
| P-11485 | [(CH₃—(CH₂)₁₀CO-Ada)-Tyr¹, D-Arg², Ala⁴, Cpa⁶, Ala⁸, Har⁹, |
| | Tyr(Me)¹⁰, His¹¹, Orn¹², Abu¹⁵, His²⁰, Orn²¹, Nle²⁷, D-Arg²⁸, |
| | Har²⁹-Ada]hGH-RH(1-29)NH₂ |
| P-11487 | [(CH₃—(CH₂)₁₀CO-Ada)-Tyr¹, D-Arg², Ala⁴, Cpa⁶, Ala⁸, Har⁹, |
| | Tyr(Me)¹⁰, His¹¹, Orn¹², Abu¹⁵, His²⁰, Orn²¹, Nle²⁷, D-Arg²⁸, |
| | Har²⁹]hGH-RH(1-29)NH₂ |
| P-11491 | [(Dca-Ada)-Tyr¹, D-Arg², Ala⁴, (Phe(F)₅)⁶, Ala⁸, Har⁹, |
| | Tyr(Me)¹⁰, His¹¹, Orn¹², Abu¹⁵, His²⁰, Orn²¹, Nle²⁷, D-Arg²⁸, |
| | Har²⁹]hGH-RH(1-29)NH₂ |
| P-11497 | [(Ac-Amc)-Tyr¹, D-Arg², Ala⁴, Cpa⁶, Ala⁸, Har⁹, Tyr(Me)¹⁰, |
| | His¹¹, Orn¹², Abu¹⁵, His²⁰, Orn²¹, Nle²⁷, D-Arg²⁸, Har²⁹- |
| | Ada³⁰]hGH-RH(1-30)NH₂ |
| P-11499 | [PhAc-Tyr¹, D-Arg², Ala⁴, (Phe(F)₅)⁶ , Ala⁸, Har⁹, Tyr(Me)¹⁰, |
| | His¹¹, Orn¹², Abu¹⁵, His²⁰, Orn²¹, Nle²⁷, D-Arg²⁸, Har²⁹- |
| | Ada]hGH-RH(1-29)NH₂ |
| P-11501 | [(Ac-Ada)-Tyr¹, D-Arg², Ala⁴, Cpa⁶, Ala⁸, Har⁹, Tyr(Me)¹⁰, |
| | His¹¹, Orn¹², Abu¹⁵, His²⁰, Orn²¹, Nle²⁷, D-Arg²⁸, Har²⁹-β-Ala- |
| | Lys(Oct)]hGH-RH(1-29)NH₂ |
| P-11503 | [(Dca-Ada)-Tyr¹, D-Arg², Ala⁴, Cpa⁶, Ala⁸, Har⁹, Tyr(Me)¹⁰, |
| | His¹¹, Orn¹², Abu¹⁵, His²⁰, Orn²¹, Nle²⁷, D-Arg²⁸, Har²⁹-β-Ala- |
| | Lys(Oct)]hGH-RH(1-29)NH₂ |
| P-11513 | [Dca-Tyr¹, D-Arg², Ala⁴, Cpa⁶, Ala⁸, Har⁹, Tyr(Me)¹⁰, His¹¹, |
| | Orn¹², Abu¹⁵, Har²⁰, Orn²¹, Nle²⁷, D-Arg²⁸, Har²⁹]hGH-RH(1-29) |
| | NH₂ |
| P-11515 | [(Dca-Ada)-Tyr¹, D-Arg², Ala⁴, Cpa⁶, Ala⁸, Har⁹, Tyr(Me)¹⁰, |
| | His¹¹, Lys(0-11)¹², Abu¹⁵, His²⁰, Orn²¹, Nle²⁷, D-Arg²⁸, |
| | Har²⁹]hGH-RH(1-29)NH₂ where (0-11) denotes the following |
| | peptide sequence: PhAc-Tyr-D-Arg-Asp-Ala-Ile-Cpa-Thr- |
| | Ala-Har-Tyr(Me)-His-; and the C-terminal carbonyl group of |
| | the (0-11) peptide sequence forms an amide bond with the |
| | epsilon amino group of Lys¹² |
| P-11521 | [(Dca-Amc)-Tyr¹, D-Arg², Ala⁴, Cpa⁶, Ala⁸, Har⁹, Tyr(Me)¹⁰, |
| | His¹¹, Orn¹², Orn¹⁵, His²⁰, Orn²¹, Nle²⁷, D-Arg²⁸, Har²⁹- |
| | Ada]hGH-RH(1-29)NH₂ |
| P-11523 | [PhAc-Tyr¹, D-Arg², Ala⁴, Cpa⁶, Ala⁸, Har⁹, Tyr(Me)¹⁰, His¹¹, |
| | Orn¹², Orn¹⁵, Glu¹⁷, His²⁰, Orn²¹, Nle²⁷, D-Arg²⁸, Har²⁹- |
| | Ada]hGH-RH(1-29)NH₂ |
| P-11525 | [PhAc-Tyr¹, D-Arg², Ala⁴, Cpa⁶, Ala⁸, Har⁹, Tyr(Me)¹⁰, His¹¹, |
| | Orn¹², Orn¹⁵, His²⁰, Orn²¹, Nle²⁷, D-Arg²⁸, Har²⁹-Ada]hGH- |
| | RH(1-29)NH₂ |
| P-11601 | [(CH₃—(CH₂)₁₀—CO-Ada)-Tyr¹, D-Arg², Ala⁴, (Phe(F)₅)⁶, Ala⁸, |
| | Har⁹, Tyr(Me)¹⁰, His¹¹, Orn¹², Abu¹⁵, His²⁰, Orn²¹, Nle²⁷, D- |
| | Arg²⁸, Har²⁹]hGH-RH(1-29)NH₂ |
| P-11603 | [(Dca-Ada)-Tyr¹, D-Arg², Ala⁴, (Phe(F)₅)⁶, Ala⁸, Har⁹, |
| | Tyr(Me)¹⁰, His¹¹, Abu¹⁵, His²⁰, Nle²⁷, D-Arg²⁸, Har²⁹]hGH- |
| | RH(1-29)NH₂ |
| P-11606 | [(PhAc-Ada)-Tyr¹, D-Arg², Ala⁴, (Phe(F)₅)⁶, (Me-Ala)⁸, Har⁹, |
| | Tyr(Me)¹⁰, His¹², Orn¹², Abu¹⁵, His²⁰, Orn²¹, Nle²⁷, D-Arg²⁸, |
| | Har²⁹, Agm]hGH-RH(1-29) |
| P-11611 | [(Ac-Amc)-Tyr¹, D-Arg², Ala⁴, (Phe(F)₅)⁶, Ala⁸, Har⁹, |
| | Tyr(Me)¹⁰, His¹¹, Orn¹², Abu¹⁵, His²⁰, Orn²¹, Nle²⁷, D-Arg²⁸, |
| | Har²⁹-Ada]hGH-RH(1-29)NH₂ |
| P-11612 | [(Ac-Ada)-Tyr¹, D-Arg², Ala⁴, (Phe(F)₅)⁶, Ala⁸, Har^{9,} |
| | Tyr(Me)¹⁰, His¹¹, Orn¹², Abu¹⁵, His²⁰, Orn²¹, Nle²⁷, D-Arg²⁸, |
| | Har²⁹-Ada]hGH-RH(1-29)NH₂ |
| P-11620 | [(Ac-Amc)-Tyr¹, D-Arg², Ala⁴, Cpa⁶, Ala⁸, Har⁹, Tyr(Me)¹⁰, |
| | His¹¹, Abu¹⁵, His²⁰, Nle²⁷, D-Arg²⁸, Arg²⁹-Ada]hGH-RH(1-29)NH₂ |
| P-11621 | [(Me-NH-Sub)-Tyr¹, D-Arg², Ala⁴, Cpa⁶, Ala⁸, Har⁹, |
| | Tyr(Me)¹⁰, His¹¹, Abu¹⁵, His²⁰, Nle²⁷, D-Arg²⁸, Har²⁹- |
| | Ada]hGH-RH(1-29)NH₂ |
| P-11630 | [(Ac-Ada)-Tyr¹, D-Arg², Ala⁴, Cpa⁶, (Me-Ala)⁸, Har⁹, |
| | Tyr(Me)¹⁰, His¹¹, Abu¹⁵, His²⁰, Nle²⁷, D-Arg²⁸, Har²⁹- |
| | Amc]hGH-RH(1-29)NH₂ |
| P-11701 | [(Dca-Ada)-Tyr¹, D-Arg², Ala⁴, Cpa⁶, Ala⁸, Har⁹, Tyr(Me)¹⁰, |
| | His¹¹, Orn¹², Abu¹⁵, Glu¹⁷, Orn²¹, Nle²⁷, D-Arg²⁸, Har²⁹- |
| | AE₄P]hGH-RH(1-29)NH₂ |
| P-11702 | [(Dca-Ada)-Tyr¹, D-Arg², Ala⁴, Cpa⁶, Ala⁸, Har⁹, Tyr(Me)¹⁰, |
| | His¹¹, Orn¹², Abu¹⁵, Glu¹⁷, His²⁰, Orn²¹, Nle²⁷, D-Arg²⁸, Har²⁹- |
| | AE₄P]hGH-RH(1-29)NH₂ |
| P-11703 | [(Dca-Ada)-Tyr¹, D-Arg², Ala⁴, Cpa⁶, Ala⁸, Har⁹, Tyr(Me)¹⁰, |
| | His¹¹, Orn¹², Abu¹⁵, Glu¹⁷, Har²⁰, Orn²¹, Nle²⁷, D-Arg²⁸, |
| | Har²⁹-AE₄P]hGH-RH(1-29)NH₂ |
| P-11704 | [(CH₃—(CH₂)₁₀—CO-Ada)-Tyr¹, D-Arg², Ala⁴, Cpa⁶, Ala⁸, Har⁹, |
| | Tyr(Me)¹⁰, His¹¹, Orn¹², Abu¹⁵, His²⁰, Orn²¹, Me²⁷, D-Arg²⁸, |
| | Har²⁹-AE₄P]hGH-RHG-29)NH₂ |

The peptides are synthesized by suitable methods such as by exclusive solid phase techniques, by partial solid-phase techniques, by fragment condensation or by classical solution phase synthesis. For example, the techniques of exclusive solid-phase synthesis are set forth in the textbook “Solid Phase Peptide Synthesis”, J. M. Stewart and J. D. Young, Pierce Chem. Company, Rockford, Ill., 1984 (2nd. ed.), and M. Bodanszky, “Principles of Peptide Synthesis”, Springer Verlag, 1984. The hGHRH antagonist peptides are preferably prepared using solid phase synthesis, such as that generally described by Merrifield, J. Am. Chem. Soc, 85 p. 2149 (1963), although other equivalent chemical syntheses known in the art can also be used as previously mentioned.

The synthesis is carried out with amino acids that are protected at their alpha amino group. Urethane type protecting groups (Boc or Fmoc) are preferably used for the protection of the alpha amino group. In certain cases, protected omega-amino acids are also used during the synthesis. Boc or Fmoc protecting groups are also appropriate for the protection of omega-amino groups.

In solid phase synthesis, the N-alpha-protected or N-omega-protected amino acid moiety which forms the aminoacyl group of the final peptide at the C-terminus is attached to a polymeric resin support via a chemical link. After completion of the coupling reaction, the alpha (or omega) amino protecting group is selectively removed to allow subsequent coupling reactions to take place at the amino-terminus, preferably with 50% TFA in DCM when the N-alpha-(N-omega-) protecting group is Boc, or by 20% piperidine in DMF when the N-alpha-(N-omega-) protecting group is Fmoc. The remaining amino acids with similarly Boc or Fmoc-protected alpha (or omega) amino groups are coupled stepwise to the free amino group of the preceding amino acid on the resin to obtain the desired peptide sequence. Because the amino acid residues are coupled to the alpha (or omega) amino group of the C-terminus residue, growth of the synthetic hGHRH analogue peptides begins at the C terminus and progress towards the N-terminus. When the desired sequence has been obtained, the peptide is acylated, or the amino group is left free at the N-terminus, and the peptide is removed from the support polymer.

Each protected amino acid is used in excess (2.5 or 3 equivalents) and the coupling reactions are usually carried out in DCM, DMF or mixtures thereof. The extent of completion of the coupling reaction is monitored at each stage by the ninhydrin reaction. In cases where incomplete coupling is determined, the coupling procedure is repeated, or a capping by acetylation of unreacted amino groups is carried out, before removal of the alpha (or omega) amino protecting group prior to the coupling of the next amino acid. Additional synthesis and purification procedures have been disclosed in U.S. patent application Ser. No. 12/890,626 which is incorporated herein by reference in its entirety.

The peptides disclosed herein may be used for treating Alzheimer's disease and other neurodegenerative disorders In some embodiments, a method of treating a subject having Alzheimer's disease may involve administering to the subject a therapeutic amount of growth hormone releasing hormone (GHRH) peptide antagonist. In some embodiments, the GHRH antagonist peptide may be formula I or formula II. In some embodiments, the GHRH antagonist peptide may be one or more of peptides listed in paragraph [0046].

In some embodiments, a method for treating a subject having amyloid plaque deposits may involve administering to the subject a therapeutic amount of growth hormone releasing hormone (GHRH) peptide antagonist. In some embodiments, the GHRH antagonist peptide may be formula I or formula II. In some embodiments, the GHRH antagonist peptide may be one or more of peptides listed in paragraph [0046].

In some embodiments, a method of treating a subject having a neurodegenerative disease may involve administering to the subject a therapeutic amount of growth hormone releasing hormone (GHRH) peptide antagonist. The neurodegenerative disease may be Alzheimer's disease, senile dementia, dementia with Lewy Bodies, Parkinson's disease, Huntington's disease, amyotrophic lateral sclerosis, and any combination thereof. In some embodiments, the neurodegenerative diseases may also be Mediterranean fever, Muckle-Wells syndrome, idiopathic myeloma, amyloid polyneuropathy, amyloid cardiomyopathy, systemic neuritic amyloidosis, amyloid polyneuropathy, hereditary cerebral hemorrhage with amyloidosis, Down's syndrome, Scrapie, Creutzfeldt-Jacob disease, Kuru, Gerstamnn-Straussler-Scheinker syndrome, medullary carcinoma of the thyroid, isolated atrial amyloid, β2-microglobulin amyloid in dialysis patients, inclusion body myositis, β2-amyloid deposits in muscle wasting disease, type II diabetes, and combinations thereof.

In some embodiments, the GHRH antagonist peptide may be formula I or formula II. In some embodiments, the GHRH antagonist peptide may be one or more of peptides listed in paragraph [0046].

In some embodiments, a method of protecting neuronal cells from oxidative stress may involve contacting the neuronal cells with a growth hormone releasing hormone (GHRH) peptide antagonist. In some embodiments, a method of improving viability of neuronal cells may involve contacting the neuronal cells with a growth hormone releasing hormone (GHRH) peptide antagonist. In some embodiments, the GHRH antagonist peptide may be formula I or formula II. In some embodiments, the GHRH antagonist peptide may be one or more of peptides listed in paragraph [0046]. In the above methods described herein, the method may be in vivo or in vitro. In some embodiments, the local concentration of the GHRH peptide antagonist used may be from about 1 nM to about 100 mM, about 1 nM to about 10 mM, about 1 nM to about 1 mM, about 1 nM to about 0.1 mM, or about 1 nM to about 10 nM.

In some embodiments, the GHRH peptide antagonist may be administered with other therapeutic agents in combination to treat Alzheimer's disease and other neurodegenerative disorders. Non-limiting examples of such therapeutic agents include a NMDA receptor antagonist, an inhibitor of amyloid AB peptide, a phosphodiesterase (PDE5) inhibitor, a PDE4 inhibitor, a monoamine oxidase inhibitor, a VEGF protein, a trophic growth factor, a HIF activator, a HIF prolyl A-hydroxylases inhibitor, an anti-apoptotic compound, an activity-dependent neurotrophic protein (ADNP) agonist, an activity-dependent neurotrophic factor (ADNF) agonist, an activator of an AMPA-type glutamate receptor, a serotonin 5-HT1A receptor agonist, a serotonin IA receptor antagonist, a nicotinic alpha-7 receptor agonist, a neuronal L-type calcium channel modulator, a 5-HT4 receptor agonist, an anti-inflammatory agent, and a pharmaceutically acceptable salt thereof. In some embodiments, the GHRH peptide antagonist may be co-administered, concurrently administered, or sequentially administered with at least one of the therapeutic agents.

The peptides of the invention may be administered in the form of pharmaceutically acceptable, nontoxic salts, such as acid addition salts. Illustrative of such acid addition salts are hydrochloride, hydrobromide, sulphate, phosphate, fumarate, gluconate, tannate, maleate, acetate, trifluoroacetate, citrate, benzoate, succinate, alginate, pamoate, malate, ascorbate, tartarate, and the like. Particularly preferred antagonists are salts of low solubility, e.g., pamoate salts and the like. These exhibit long duration of activity.

Formulations containing the peptides of the present invention and a suitable carrier can be solid dosage forms which include, but are not limited to, softgels, tablets, capsules, cachets, pellets, pills, powders and granules; topical dosage forms which include, but are not limited to, solutions, powders, fluid emulsions, fluid suspensions, semi-solids, ointments, pastes, creams, gels and jellies, and foams; and parenteral dosage forms which include, but are not limited to, solutions, suspensions, emulsions, and dry powder; comprising an effective amount of a polymer or copolymer of the present invention. In some embodiments, a single dose may comprise one or more softgels, tablets, capsules, cachets, pellets, pills, or the like. Specific examples include, for example, a dose comprising 1, 2, 3, or 4 softgels, tablets, capsules, cachets, pellets, pills or the like.

In some embodiments, one or more softgels, tablets, capsules, cachets, pellets, pills, or the like can be taken to achieve the desired dosing. In some embodiments, one or more softgels, tablets, capsules, cachets, pellets, pills, or the like can be taken simultaneously to achieve the desired dosing. In yet another embodiment one or more softgels, tablets, capsules, cachets, pellets, pills, or the like can be taken separately during the course of a specified time period such as for example, a 24 hour period. For example, one or more softgels, tablets, capsules, cachets, pellets, pills, or the like can be taken twice in a 24 hour period to achieve the desired dose. In some embodiments, one or more softgels, tablets, capsules, cachets, pellets, pills, or the like can be taken with a meal. For example one or more softgels, tablets, capsules, cachets, pellets, pills, or the like can be taken with each meal during the course of a 24 hour period to achieve the desired dose.

It is also known in the art that the active ingredients can be contained in such formulations with pharmaceutically acceptable diluents, fillers, disintegrants, binders, lubricants, surfactants, hydrophobic vehicles, water soluble vehicles, emulsifiers, buffers, humectants, moisturizers, solubilizers, preservatives and the like. The means and methods for administration are known in the art and an artisan can refer to various pharmacologic references for guidance. For example, Modern Pharmaceutics, Banker & Rhodes, Marcel Dekker, Inc. (1979); and Goodman & Gilman's The Pharmaceutical Basis of Therapeutics, 6th Edition, MacMillan Publishing Co., New York (1980) can be consulted.

In some embodiments, the pharmaceutical excipient may include, without limitation, binders, coating, disintegrants, fillers, diluents, flavors, colors, lubricants, glidants, preservatives, sorbents, sweeteners, conjugated linoleic acid (CLA), gelatin, beeswax, purified water, glycerol, any type of oil, including, without limitation, fish oil or soybean oil, or the like. Pharmaceutical compositions of the peptides/compounds also can comprise suitable solid or gel phase carriers or excipients. Examples of such carriers or excipients include but are not limited to calcium carbonate, calcium phosphate, various sugars, starches, cellulose derivatives, gelatin, and polymers such as, e.g., polyethylene glycols.

The peptides/compounds of the present invention can be administered in the conventional manner by any route where they are active. Administration can be systemic, parenteral, topical, or oral. For example, administration can be, but is not limited to, parenteral, such as subcutaneous, intramuscular, intraperitoneal, intracavity, intrathecal, transdermal, and intravenous. Oral, buccal, or ocular routes, intravaginal, inhalation, depot injections, or implants may also be used to deliver the peptides. Thus, modes of administration for the peptides/compounds of the present invention (either alone or in combination with other pharmaceuticals) can be, but are not limited to, sublingual, injectable (including short-acting, depot, implant and pellet forms injected subcutaneously or intramuscularly), or by use of vaginal creams, suppositories, pessaries, vaginal rings, rectal suppositories, intrauterine devices, and transdermal forms such as patches and creams.

Alternatively, the peptides may be administered as an intranasal spray with an appropriate carrier or by pulmonary inhalation. One suitable route of administration is a depot form formulated from a biodegradable suitable polymer, e.g., poly-D,L-lactide-coglycolide as microcapsules, microgranules or cylindrical implants containing dispersed antagonistic compounds.

Specific modes of administration will depend on the indication. The selection of the specific route of administration and the dose regimen is to be adjusted or titrated by the clinician according to methods known to the clinician in order to obtain the optimal clinical response. The amount of peptides/compounds to be administered is that amount which is therapeutically effective. The dosage to be administered will depend on the characteristics of the subject being treated, e.g., the particular animal or human being treated, age, weight, health, types of concurrent treatment, if any, and frequency of treatments, and can be easily determined by one of skill in the art (e.g., by the clinician).

The amount of peptide needed depends on the type of pharmaceutical composition and on the mode of administration. In some embodiments, the GHRH peptide antagonist administration may be at a dosage of about 0.005 mg/kg/dose to about 100 mg/kg/dose, about 0.005 mg/kg/dose to about 10 mg/kg/dose, about 0.005 mg/kg/dose to about 1 mg/kg/dose, about 0.005 mg/kg/dose to about 0.5 mg/kg/dose, about 0.005 mg/kg/dose to about 0.1 mg/kg/dose, or about 0.005 mg/kg/dose to about 0.05 mg/kg/dose. In cases where human subjects receive solutions of GHRH antagonists, administered by i.m. or s.c. injection, or in the form of intranasal spray or pulmonary inhalation, the typical doses are between 2-20 mg/day/patient, given once a day or divided into 2-4 administrations/day. When the GHRH antagonists are administered intravenously to human patients, typical doses are in the range of 8-80 μg/kg of body weight/day, divided into 1-4 bolus injections/day or given as a continuous infusion. When depot preparations of the GHRH antagonists are used, e.g. by i.m. injection of pamoate salts or other salts of low solubility, or by i.m. 10 or s.c. administration of microcapsules, microgranules, or implants containing the antagonistic compounds dispersed in a biodegradable polymer, the typical doses are between 1-10 mg antagonist/day/patient.

The peptides/compounds of the present invention can be formulated for parenteral administration by injection, e.g., by bolus injection or continuous infusion. The peptides/compounds can be administered by continuous infusion subcutaneously over a period of about 15 minutes to about 24 hours. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents.

For oral administration, the peptides/compounds can be formulated readily by combining these peptides/compounds with pharmaceutically acceptable carriers well known in the art. Such carriers enable the peptides/compounds of the invention to be formulated as tablets, pills, dragees, capsules, liquids, gels, syrups, slurries, suspensions and the like, for oral ingestion by a patient to be treated. Pharmaceutical preparations for oral use can be obtained by adding a solid excipient, optionally grinding the resulting mixture, and processing the mixture of granules, after adding suitable auxiliaries, if desired, to obtain tablets or dragee cores. Suitable excipients include, but are not limited to, fillers such as sugars, including, but not limited to, lactose, sucrose, mannitol, and sorbitol; cellulose preparations such as, but not limited to, maize starch, wheat starch, rice starch, potato starch, gelatin, gum tragacanth, methyl cellulose, hydroxypropylmethyl-cellulose, sodium carboxymethylcellulose, and polyvinylpyrrolidone (PVP). If desired, disintegrating agents can be added, such as, but not limited to, the cross-linked polyvinyl pyrrolidone, agar, or alginic acid or a salt thereof such as sodium alginate.

Dragee cores can be provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active peptides/compound doses.

Pharmaceutical preparations which can be used orally include, but are not limited to, push-fit capsules made of gelatin, as well as soft, sealed capsules made of gelatin and a plasticizer, such as glycerol or sorbitol. The push-fit capsules can contain the active ingredients in admixture with filler such as, e.g., lactose, binders such as, e.g., starches, and/or lubricants such as, e.g., talc or magnesium stearate and, optionally, stabilizers. In soft capsules, the active peptides/compounds can be dissolved or suspended in suitable liquids, such as fatty oils, liquid paraffin, or liquid polyethylene glycols. In addition, stabilizers can be added. All formulations for oral administration should be in dosages suitable for such administration.

For buccal administration, the compositions can take the form of, e.g., tablets or lozenges formulated in a conventional manner.

For administration by inhalation, the compositions for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of, e.g., gelatin for use in an inhaler or insufflator can be formulated containing a powder mix of the peptides/compound and a suitable powder base such as lactose or starch.

The compositions of the present invention can also be formulated in rectal compositions such as suppositories or retention enemas, e.g., containing conventional suppository bases such as cocoa butter or other glycerides.

In addition to the formulations described previously, the compositions of the present invention can also be formulated as a depot preparation. Such long acting formulations can be administered by implantation (for example subcutaneously or intramuscularly) or by intramuscular injection.

Depot injections can be administered at about 1 to about 6 months or longer intervals. Thus, for example, the peptides/compounds can be formulated with suitable polymeric or hydrophobic materials (for example as an emulsion in an acceptable oil) or ion exchange resins, or as sparingly soluble derivatives, for example, as a sparingly soluble salt.

In transdermal administration, the compositions of the present invention, for example, can be applied to a plaster, or can be applied by transdermal, therapeutic systems that are consequently supplied to the organism.

The compositions of the present invention can also be administered in combination with other active ingredients, such as, for example, adjuvants, protease inhibitors, or other compatible drugs or compounds where such combination is seen to be desirable or advantageous in achieving the desired effects of the methods described herein.

In some embodiments, the disintegrant component comprises one or more of croscarmellose sodium, carmellose calcium, crospovidone, alginic acid, sodium alginate, potassium alginate, calcium alginate, an ion exchange resin, an effervescent system based on food acids and an alkaline carbonate component, clay, talc, starch, pregelatinized starch, sodium starch glycolate, cellulose floc, carboxymethylcellulose, hydroxypropylcellulose, calcium silicate, a metal carbonate, sodium bicarbonate, calcium citrate, or calcium phosphate.

In some embodiments, the diluent component comprises one or more of mannitol, lactose, sucrose, maltodextrin, sorbitol, xylitol, powdered cellulose, microcrystalline cellulose, carboxymethylcellulose, carboxyethylcellulose, methylcellulose, ethylcellulose, hydroxyethylcellulose, methylhydroxyethylcellulose, starch, sodium starch glycolate, pregelatinized starch, a calcium phosphate, a metal carbonate, a metal oxide, or a metal aluminosilicate.

In some embodiments, the optional lubricant component, when present, comprises one or more of stearic acid, metallic stearate, sodium stearyl fumarate, fatty acid, fatty alcohol, fatty acid ester, glyceryl behenate, mineral oil, vegetable oil, paraffin, leucine, silica, silicic acid, talc, propylene glycol fatty acid ester, polyethoxylated castor oil, polyethylene glycol, polypropylene glycol, polyalkylene glycol, polyoxyethylene-glycerol fatty ester, polyoxyethylene fatty alcohol ether, polyethoxylated sterol, polyethoxylated castor oil, polyethoxylated vegetable oil, or sodium chloride.

This invention and embodiments illustrating the method and materials used may be further understood by reference to the following non-limiting examples. The present invention is described in connection with the following examples which are set forth for the purposes of illustration only. In the examples, optically active protected amino acids in the L-configuration are used except where specifically noted.

### EXAMPLES

### Example 1

### The Aqueous Solution for Intramuscular Injection

Phac-Ada-Tyr¹-D-Arg²-Asp³-Ala⁴-Ile⁵-Phe(F)₅⁶-Thr⁷-Ala⁸-Har⁹-Tyr(Me)¹⁰-His¹¹-Orn¹²-Val¹³-Leu¹⁴-Abu¹⁵-Gln¹⁶-Leu¹⁷-Ser¹⁸-Ala¹⁹-His²⁰-Orn²¹-Leu²²-Leu²³-Gln²⁴-Asp²⁵-Ile²⁶-Nle²⁷-D-Arg²⁸-Har²⁹-NH₂ (MIA-602) 500.0 mg

Gelatin, nonantigenic 5.0 mg

Water for injection q.s. ad 100.0 mL

The gelatin and GH-RH antagonist Peptide MIA-602 are dissolved in water for injection, and then the solution is sterile filtered.

### Example 2

### Long Acting Intramuscular Injectable Formulation (Sesame Oil Gel)

Phac-Ada-Tyr¹-D-Arg²-Asp³-Ala⁴-Ile⁵-Phe(F)₅⁶-Thr⁷-Ala⁸-Har⁹-Tyr(me)¹⁰-His¹¹-Orn¹²-Val¹³-Leu¹⁴-Abu¹⁵-Gln¹⁶-Leu¹⁷-Ser¹⁸-Ala¹⁹-His²⁰-Orn²¹-Leu²²-Leu²³-Gln²⁴-Asp²⁵-Ile²⁶-Nle²⁷-D-Arg²⁸-Har²⁹-Agm-NH₂ (MIA-604).

Aluminum monostearate, USP 20.0 mg

Sesame oil q.s. 1.0 mL

The aluminum monostearate is combined with the sesame oil and heated to 125° C. with stirring until a clear yellow solution forms. This mixture is then autoclaved for sterility and allowed to cool. The GHRH antagonist Peptide MIA-604 is then added aseptically with trituration. Particularly preferred antagonists are salts of low solubility, e.g., pamoate salts and the like. These exhibit long duration of activity.

### Example 3

### Long Acting Intramuscular (IM) Injectable-Biodegradable Polymer Microcapsules

Microcapsules are made from the following:

25/75 glycolide/lactide copolymer (0.5 intrinsic viscosity) 99%
Phac-Ada-Tyr¹-D-Arg²-Asp³-Ala⁴-Ile⁵-Phe(F)₅⁶-Thr⁷-Me-Ala⁸-Har⁹-Tyr(Me)¹⁰-His¹¹-Orn¹²-Val¹³-Leu¹⁴-Abu¹⁵-Gln¹⁶-Leu¹⁷-Ser¹⁸-Ala¹⁹-His²⁰-Orn²¹-Leu²²-Leu²³-Gln²⁴-Asp²⁵-Ile²⁶-Nle²⁷-D-Arg²⁸-Har²⁹-Agm-NH₂ (MIA-606) 1%.

25 mg of the above microcapsules are suspended in 1.0 mL of the following vehicle:

| | |
|---|---|
| Dextrose | 5.0% |
| CMC, sodium | 0.5% |
| Benzyl alcohol | 0.9% |
| Tween 80 | 0.1% |
| Water, purified q.s. | 93.5% |

### Example 4

### Use of GHRH Antagonist Peptides in Different Models of Alzheimer's Disease

Methods

For treatment, MIA-690 was dissolved in an aqueous solution of 0.1% DMSO (Sigma) and 10% propylene glycol (Sigma-Aldrich, St. Louis, Mo.).

Transgenic mice (5XFAD strain) were obtained from The Jackson Laboratories (Bar Harbor, Me.). The animals were housed in sterile cages in a temperature-controlled room with a 12-h light/12-h dark schedule and were fed with autoclaved chow and water, ad libitum. Both sexes were used, evenly distributed between the different treatment groups. For the Morris water maze experiments 41 adult mice (approximately 3 months old) were used and were divided into 4 treatment groups, each of which received the following subcutaneous daily treatment for 6 months: group 1: (control), vehicle solution; group 2: MIA-690 (2 μg); group 3: MIA-690 (5 μg); and group 4: MIA-690 (10 μg).

The maze was a circular white steel pool (120 cm diameter; 40 cm high). The pool was filled to approximately 30 cm with water at room temperature (22±2° C.). The water was made opaque with non-toxic white liquid tempura paint (Crayola, Easton, Pa.). The black-furred animals provided sufficient contrast for video tracking The experiment was performed according to the guidelines of the literature. The pool was divided virtually into four quadrants with four equidistant release points around the edge. The release points were labeled according to the points of compass: south (S), west (W), north (N), east (E). The goal platform (10 cm diameter) was submerged 1.5 cm beneath the water surface in the center of one of the quadrants (at 30 cm radial distance from the rim of the pool). The platform positions were labeled according to the nomenclature of the recording software (Water Maze Software, Columbus Instruments, Columbus, Ohio) and the compass directions: 1, north-west (NW); 2, south-west (SW); 3, south-east (SE); 4, north-east (NE). For each training trial, a mouse was released at a semi-randomly assigned release point and allowed to swim freely. Once the platform was reached, the mouse was allowed to remain there for 15 s. If the platform was not located after 60 s, the mouse was gently guided to the platform and allowed to remain there for 15 s. Trials of individuals were separated by about 70-80 min (one trial lasted for almost 2 min) and the mice were dried between trials to prevent hypothermia. Differences in swimming speed, motivation and tendency to float were first assessed during 2 preliminary sessions, when the platform was visible; and “floaters” were excluded from the study. During a daily session, each animal then received a block of 4 training trials with the platform hidden; one complete training cycle consisted of 5 consecutive days. The platform remained in the same quadrant for all acquisition trials and for all mice. At the end of the training, on the 6th day, a probe test was completed wherein the platform was removed from the pool and each mouse was allowed to search freely for 60 s. A video-tracking system was used to monitor and quantify performance (Videomex-One hardware and Water Maze Software, Columbus Instruments, Columbus, Ohio). For all trials, peripheral cues around the maze environs remained constant throughout testing. The observed and recorded parameters for the trainings were: escape latency, path length, cumulative distance (CD) and proximity average (PA), while for probes CD, PA, platform crossings (PC), entries to platform quadrant (EPQ), path length in platform quadrant (PPQ) and time spent in platform quadrant (TPQ) were used. Averages of the output variables of the four individual trials were used for comparison and statistical evaluation. Mice were followed monthly for 6 months (between the age of 3 and 9 months) after commencing their treatment. In addition to the survival of mice, their training and probe values were recorded monthly. For comparison of the probe values, the CD, the PC, the EPQ, the PPQ and the TPQ were used but the change in escape latency between the first and the fifth day was also found to be a sensitive marker. Between the monthly sessions the probe sessions facilitated extinction of memory and for the next training session the platform was semi-randomly relocated to a new position.

At the end of the experiment, the mice were sacrificed by cervical dislocation and decapitation, necropsy was performed, and the brains were removed. The hemispheria were immediately snap-frozen in liquid nitrogen and stored at 80° C. for PCR and proteomic studies. For the determination of amyloid-β₁₋₄₂ and total T-protein levels, mouse-specific ELISA kits were used according to the manufacturer's instructions (Invitrogen, Carlsbad, Calif.).

HCN-2 cells (American Type Culture Collection, Manassas, Va., USA) were cultured in DMEM medium (supplemented with 10% Fetal Bovine Serum (FBS) and 0.1% penicillin/streptomycin) at 37° C. and in an atmosphere of air and 5% CO₂. At 70-80 percent of confluency, the cultures were trypsinized and re-suspended in fresh serum-containing medium and either transferred into T-75 flasks or directly plated into 48-well micro plates at 10,000 cells/cm². The next day, the differentiation of HCN-2 cells was induced by adding fresh medium containing 25 ng/ml NGF, 0.5 mM dibutyryl cAMP and 0.1 mM isobutylmethylxanthine (IBMX) (all from Sigma-Aldrich, St. Louis, Mo.) for a week. Human amyloid-β₁₋₄₂ (Abbiotec LLc, San Diego, Calif.) stock solution (10 mM) was prepared in DMSO and immediately diluted to appropriate concentrations in the assay medium. The medium used for neurotoxicity assay was N2-supplemented DMEM/F12 (Gibco BRL, NY) with 10% FBS. The treatment groups were control, amyloid-β₁₋₄₂, and the combination treatments with amyloid-β₁₋₄₂ and the 3 doses (10 nM, 100 nM and 1 μM) of MIA-690; controls received propylene glycol and DMSO containing medium. The effect of the analog on proteo-toxicity was evaluated after three days of exposure. The viability of the cells was determined by using the 3-(4,5-Dimethylthiazol-2-yl)-2,5-diphenyltetrazolium bromide (MTT) assay (Cell Titer Non-Radioactive Cell Proliferation Assay, Promega, Madison, Wis.), according to the manufacturer's instructions. APF assay (Invitrogen) was used for the detection of free radical formation according to the manufacturer's instructions. Concentrations of the specific proteins (IGF-I, IGF-II, GPx1, SOD1, BDNF) in the media were determined using appropriate ELISA kits according to the manufacturer's instructions. IGF-I, IGF-II and BDNF human ELISA kits were obtained from AbCam Inc. (Cambridge, Mass.) while GPx1 and SOD1 kits from Abfrontier through Biovendor, LLC, (Candler, N.C.). Readings were normalized to protein concentrations as determined by NanoDrop (NanoDrop Technologies Inc., Wilmington, Del.).

Total RNA was isolated and DNAse treated from representative hemispheria using NucleoSpin kit according to the manufacturer's instructions (Macherey-Nagel Inc., Bethlehem, Pa.). Five samples each from the control and the 10 μg MIA-690 group were analyzed. The yield and the quality of RNA samples were determined spectrophotometrically using 260 nm, and 260/280 and 260/230 nm ratio. The synthesis of cDNA was performed as follows. Briefly, 1 μg of RNA from each sample was reverse-transcribed into cDNA by RT First Strand kit (Qiagen). Reverse transcription was done in a Veriti 96-well thermal cycler (Applied Biosystems). The Mouse Alzheimer's Disease real-time quantitative PCR array (PAMM-057Z Qiagen) used in the study contains 84 unique genes related to Alzheimer's disease. All PCR arrays were performed using iQ5 Multicolor Real-Time Detections System (Bio-Rad). All genes represented by the array showed a single peak on the melting curve characteristic of the specific products. Data analysis of gene expression was performed using Excel based PCR Array Data Analysis Software provided by the manufacturer (Qiagen): fold-changes in gene expression were calculated using the ΔΔCt method and five stably expressed housekeeping genes (Act b, B2m, GAPDH, Gus b, Hsp 90 ab1) were used for normalization of the results.

Statistical evaluation of the in vivo experiments was performed by repeated measure General Linear Model (GLM) or survival analysis (Kaplan-Meier). GLM was followed by Tukey's and Fisher's post hoc tests, while for the survival analysis the Log Rank (Mantel-Cox) test was used for group-wise comparisons. The in vitro data were evaluated using t-test for independent samples or univariate GLM; the latter being followed by post hoc comparison. Results are expressed either as the means±SEM or as means and pooled standard errors (PSE)s, in the case of line plots. Differences with p<0.05, compared to the control, were considered statistically significant. Data reductions and statistical analyses were performed by SigmaPlot 12.0 (Systat Software, Inc., Chicago, Ill.) and IBM SPSS Statistics 20.0 (IBM Corporation, Armonk, N.Y.).

Results

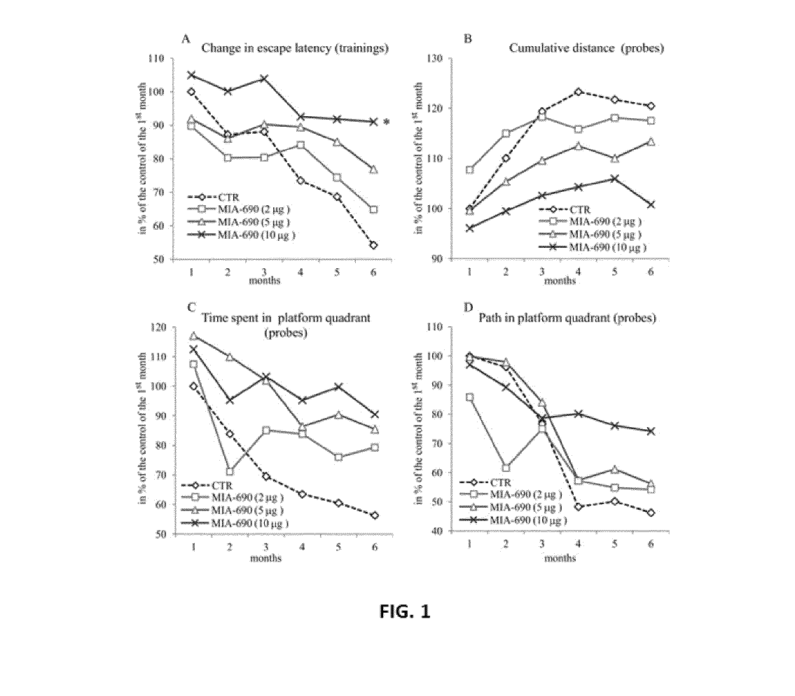

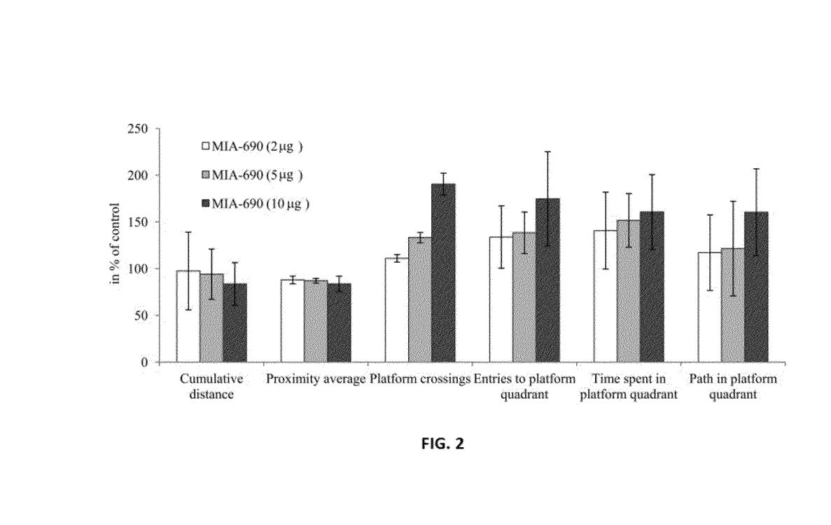

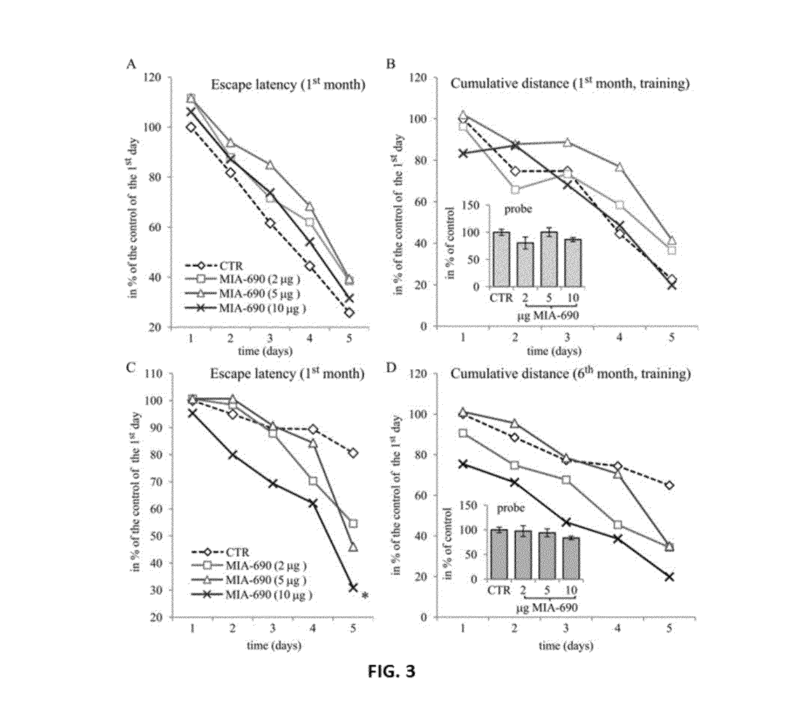

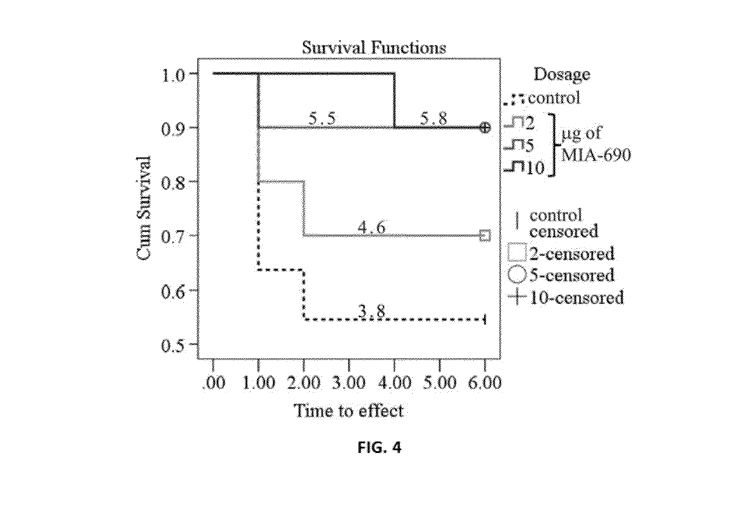

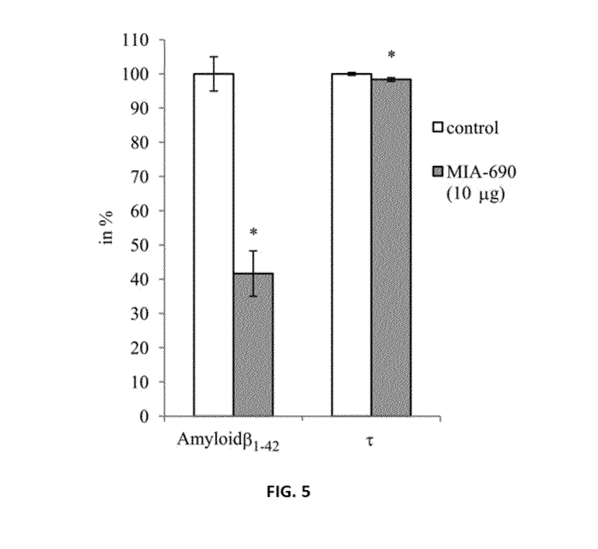

In animal studies, during the 6 month observation period, a marked deterioration of spatial learning was observed according to the probe parameters and the decrease in latency seen during the acquisition phase (FIGS. 1 A-D). Repeated measure of general linear model (GLM) revealed that 10 μg MIA-690 almost completely abolished the progressive decrease in the amplitude of the latency curve (FIG. 1A; between subject F_{3.37}=831.73, p<0.01, Tukey's post hoc test: p<0.05 vs. control). The analog also showed a tendency to attenuate the changes in other behavioral parameters (FIGS. 1 B-D and FIG. 2). After six months, conspicuous differences could be observed between the control and the MIA-690 groups during the acquisition period, especially when compared to the results of the first month (FIG. 3 A-D). The group treated with 10 μg MIA-690 performed significantly better and the effect in latency proved to be statistically significant (FIG. 1C; between subject F_{3.28}=64.37, p<0.01, Fisher's post hoc test: p<0.05 vs. control). Further, the analog appeared to prolong survival (FIG. 4). The analysis of the necropsied brain samples demonstrated that the effective concentration (10 μg) of the GHRH antagonist dramatically decreased the cerebral deposition of amyloid-β₁₋₄₂ (MIA-690 t₍₇₈₎=7.025 and p<0.05 vs. control) and slightly attenuated the total accumulation of τ-protein (MIA-690 t₍₇₈₎=2.395 and p<0.01 vs. control) in the transgenic mice (FIG. 5).

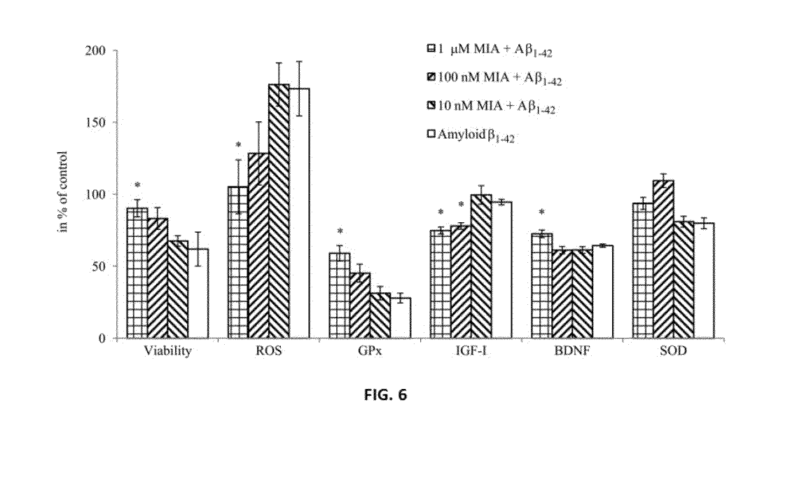

MIA-690 exerted dose-dependent effects on the viability, oxidative metabolism and mediator release of HCN-2 cells (FIG. 6). The peptide remarkably attenuated the toxic impact of co-treatment with amyloid-β₁₋₄₂ on the viability of neurons (F_{4.23}=2.8, p<0.05, Fisher's post hoc test: p<0.05, 10 μM MIA-690+amyloid-β₁₋₄₂ vs. amyloid-β₁₋₄₂) and practically abolished the generation of ROS evoked by amyloid-β₁₋₄₂ co-treatment (F_{4.43}=2.64, p<0.05, Fisher's post hoc test: p<0.05, 1 μM MIA-690+amyloid-β₁₋₄₂ vs. amyloid-β₁₋₄₂). While the analog did not have a significant and linear impact on SOD1 expression it significantly increased the glutathione-peroxidase (GPx) (F_{4.75}=15.2, p<0.01, Tukey's post hoc test: p<0.05, 1 μM MIA-690+amyloid-β₁₋₄₂ vs. amyloid-β₁₋₄₂) and brain derived neurotrophic factor (BDNF) (F_{4.75}=58.72, p<0.01, Fisher's post hoc test: p<0.01, 1 μM MIA-690+amyloid-β₁₋₄₂ vs. amyloid-β₁₋₄₂) expression at the highest applied concentration. The GHRH analog also suppressed the release of IGF-I (F_{4.75}=9.22, p<0.01), (Tukey's post hoc test: p<0.05, 100 nM MIA-690+amyloid-β₁₋₄₂ vs. amyloid-β₁₋₄₂ and p<0.01, 1 μM MIA-690+amyloid-β₁₋₄₂ vs. amyloid-β₁₋₄₂), but its effect on the secretion of IGF-II was negligible (data not presented). The PCR Array studies revealed statistically significant changes in the expression of 22 Alzheimer's disease related genes in the brain samples of the 5XFAD mice following treatment with 10 μg MIA-690 daily for six months (Table 2).

TABLE 2

| | | Fold change vs. |
|---|---|---|
| Gene | | controls |
| acetylcholinesterase | | −1.61 |
| amyloid-β (A4) precursor protein binding | family A, member 2 | −2.94 |
| | family B, member 3 | −2.04 |
| amyloid-β (A4) | precursor protein | −1.79 |
| | precursor-like protein 2 | −1.59 |
| anterior pharynx defective 1a homolog | | −1.54 |
| β-site APP-cleaving enzyme 2 (BACE2) | | −2.27 |
| caspase 4, apoptosis-related cysteine peptidase | | −3.23 |
| cyclin-dependent kinase 1 | | −2.00 |
| clusterin | | −1.72 |
| cathepsin | C | −6.67 |
| | G | −2.22 |
| growth associated protein 43 | | −1.61 |
| insulin-like growth factor 2 | | −3.22 |
| low density lipoprotein receptor-related protein 6 | | −4.76 |
| microtubule-associated protein | τ | −1.33 |
| | 2 | −1.54 |
| neural precursor cell expressed and developmentally | | −4.35 |
| down regulated gene (NEDD)8 activating | | |
| enzyme E1 subunit 1 | | |
| nicastrin | | −1.69 |
| presenilin | 1 | −2.17 |
| | 2 | −1.27 |

| | |
|---|---|
| ubiquinol cytochrome c reductase core protein 2 | 1.49 |

Discussion

Present experiments demonstrate that the GHRH antagonist, MIA-690, has several beneficial effects in each of the different models of Alzheimer's disease. Regarding the GHRH-GH-IGF axis, MIA-690 decreased the secretion of IGF-I (FIG. 6) in the supernatant of HCN-2 cell cultures. This is significant since in Alzheimer's disease one of the most important pathologic phenomena is the competition of insulin and amyloid-β for insulin-degrading enzyme (IDE). In hyperinsulinemia states, insulin, which has higher affinity to IDE, occupies the binding sites of the enzyme, rendering IDE inaccessible to amyloid-β, and causing amyloid-β accumulation. In addition to insulin, IGF-1 which is the most abundant type of the IGF family in the CNS, appears to influence cognitive decline in pathologic conditions. Decreased IGF-I signaling appears to ameliorate, directly, the amyloid-β proteotoxicity through enhancing DNA repair and increasing resistance to oxidative stress.

In the in vivo studies, the GHRH antagonist significantly and dose-dependently delayed the Alzheimer's disease-related deterioration of the acquisition phase in MWM (FIG. 1A, 3C). The peptide also tended to improve the parameters of cognitive performance by the 6^{th} month of the follow-up period as reflected by the probe values (especially the cumulative distance and platform crossings) of spatial reference memory (FIG. 1B-D, FIG. 2, FIG. 3D). The PCR Array studies (Table 2) revealed that the neuro-peptide analog, beside several possible, long-term activities, may have acute beneficial effects on learning.

The genomic and proteomic studies shed light on further possible mechanisms of action of MIA-690. The GHRH antagonist influenced the transcription of almost 2 dozen putative Alzheimer's disease markers according to the PCR Array experiments (Table 2). The most notable examples are related to the metabolism of amyloid-β, the microtubule system, apoptosis, neural signal transduction, and energy homeostasis. Regarding the metabolism of amyloid-β, the transcriptional studies revealed a remarkable inhibition of the expression of APPs and the amyloid-β precursor protein-binding proteins (APP-BP)s. Further, the GHRH antagonist decreased the transcription of the amyloid-β generating BACE2 and several components (presenilin 1, presenilin 2, anterior pharynx-defective 1 and nicastrin) of the γ-secretase complex. In the experiments described herein, substantial decreases could be detected in the “C” and “G” members of the family. The observed down-regulation of low density lipoprotein receptor-related protein (LRP)6 provide further evidence that MIA-690 can influence amyloid-β degradation, since LRPs, in cooperation with α2-macroglobulin and ApoE, are the main factors in the modulation of amyloid-β secretion/breakdown. Similar changes were observed in the expression of the microtubule-associated proteins (MAP)s. Both MAP2 and MAPτ were down-regulated. The markers of these genomic changes (amyloid-β₁₋₄₂ and total τ levels) were verified by proteomic determination. The amyloid-β₁₋₄₂ level showed especially dramatic decrease due to MIA-690 treatment (FIG. 5).

The attenuation of oxidative stress by GHRH antagonists or by the decrease in IGF-I signaling also seems to play a crucial role in the protection against amyloid-β proteotoxicity. In the experiments described herein, MIA-690 significantly decreased free radical formation (FIG. 6) of HCN-2 cells. Further, this GHRH antagonist dose-dependently augmented GPx1 levels while it did not have a significant effect on SOD1 secretion (FIG. 6). It appears that MIA-690 directly stimulates the protective antioxidant enzymes in the CNS. Therefore, present findings demonstrate that the activity of the anti-oxidative system in the MIA-690 treated animals is not only passively responsive to the free radical burden, but actively up-regulates the relevant ROS catabolic enzymes. The oxidative stress elicited by proteotoxicity inevitably leads to apoptosis. In full agreement with our anti-oxidative experiments, MIA-690 dose-dependently increased the viability of HCN-2 cells treated by amyloid-β₁₋₄₂ (FIG. 6). The GHRH antagonist also attenuated the transcription of caspase and clusterin (Table 2), both of which play important roles in apoptotic processes in the CNS. Further, GHRH antagonist treatment decreased the expression of APP-BPs and the neural precursor cell-expressed and developmentally down-regulated gene (NEDD)8 activating enzyme E1-subunit 1, which both cooperate in neddylation, one of the post-translational tagging processes that can lead to apoptosis.

Inhibition of the different levels of the GHRH-GH-IGF axis apparently exerts a beneficial impact on the progress of Alzheimer's disease. It is possible that the activation of the GHRH-GH-IGF-I axis has rejuvenating action on the cognitive performance of the elderly, similar to its somatic effects. Although, IGF-II is far less dependent on the GHRH-GH axis than IGF-I, it is important to emphasize that in the disclosed experiments, chronic peptide administration also decreased IGF-II expression in the brain samples (Table 2). Therefore, albeit acute administration of MIA-690 did not influence IGF-II secretion in HCN-2 tissue cultures, the direct effect of the peptide on memory can interfere with the beneficial actions on neurodegeneration.

The disclosure shows that peptides freely penetrate the blood-brain barrier and apparently target different levels of the pathologic cascade of Alzheimer's disease inhibiting aggregation and proteo-toxicity while restoring normal neural metabolism and regeneration.

### Example 5

### Cell Viability Assay

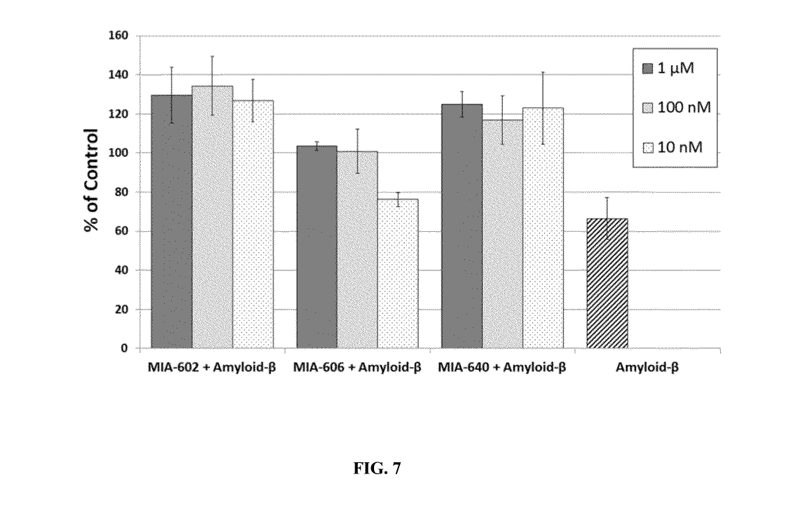

HCN-2 cells (American Type Culture Collection, Manassas, Va., USA) were cultured in DMEM medium (supplemented with 10% Fetal Bovine Serum (FBS) and 0.1% penicillin/streptomycin) at 37° C. and in an atmosphere of air and 5% CO₂. Cell were directly plated into 96-well micro plates at 5,000 cells/cm². After 7 days, to allow cell number duplication (up to 70-80% confluence), the differentiation of HCN-2 cells was induced by adding fresh medium containing 25 ng/ml NGF, 0.5 mM dibutyryl cAMP and 0.5 mM isobutylmethylxanthine (IBMX) (all from Sigma-Aldrich, St. Louis, Mo.) for a week. Then, human amyloid-β₁₋₄₂ (Sunnyvale, Calif.) stock solution (10 mM) was prepared in TRIS and then immediately diluted to appropriate concentrations in the assay medium. The medium used for neurotoxicity assay was N2-supplemented DMEMF12 (Gibco BRL, NY) with 2% FBS. The treatment groups were control, amyloid-β₁₋₄₂, and the combination treatments with amyloid-β₁₋₄₂ and 3 doses (10 nM, 100 nM and 1 μM) of the analogs (MIA-602, MIA-606 and MIA-640). The effect of the analogs on cell viability was evaluated after four days of exposure by using the 3-(4,5-Dimethylthiazol-2-yl)-2,5-diphenyltetrazolium bromide (MTT) assay (Cell Titer 96® Non-Radioactive Cell Proliferation Assay, Promega, Madison, Wis.), according to the manufacturer's instructions. All the tested peptides attenuated the toxic impact of co-treatment with amyloid-β₁₋₄₂ on the viability of neurons (FIG. 7).

1-31. (canceled)

32. A method of treating a subject having a neurodegenerative disease comprising administering to said subject a therapeutic amount of growth hormone releasing hormone (GHRH) peptide antagonist having the amino acid sequence (formula I):
R¹-Tyr¹-D-Arg²-Asp³-A⁴-Ile⁵-A⁶-Thr⁷-A⁸-Har⁹-A¹⁰-A¹¹-A¹²-Val¹³-Leu¹⁴-A¹⁵-Gln¹⁶-A¹⁷-Ser¹⁸-Ala¹⁹-A²⁰-A²¹-Leu²²-Leu²³-Gln²⁴-Asp²⁵-Ile²⁶-Nle²⁷-D-Arg²⁸-A²⁹-R²-R³-NH₂,
wherein R¹ is PhAc, Nac, Oct, N-Me-Aib, Dca, Ac-Ada, Fer, Ac-Amc, Me-NH-Sub, PhAc-Ada, Ac-Ada-D-Phe, Ac-Ada-Phe, Dca-Ada, Nac, Nac-Ada, Ada-Ada, or CH₃(CH₂)₁₀-CO-Ada;
A⁴ is Ala or Me-Ala;
A⁶ is Cpa or Phe(F)₅;
A⁸ is Ala, Pal, Dip, or Me-Ala;
A¹⁰ is FPa5,Tyr(Alk) where Alk is Me or Et;
A¹¹ is His or Arg;
A¹² is Lys, Lys(0-11), Lys(Me)₂, or Orn;
A¹⁵ is Abu or Orn;
A¹⁷ is Leu or Glu;
A²⁰ is Har or His;
A²¹ is Lys, Lys(Me)₂ or Orn;
A²⁹ is Har, Arg or Agm;
R² is β-Ala, Amc, Apa, Ada, AE₂A, AE₄P, ε-Lys(α-NH₂), Agm, or absent; and
R³ is Lys(Oct), Ahx, or absent.

33. The method of claim 32, wherein the GHRH peptide antagonist has the amino acid sequence (formula II):
R¹-Tyr¹-D-Arg²-Asp³-Ala⁴-Ile⁵-A⁶-Thr⁷-A⁸-Har⁹-A¹⁰-His¹¹-Orn¹²-Val¹³-Leu¹⁴-Abu¹⁵-Gln¹⁶-A¹⁷-Ser¹⁸-Ala¹⁹-His²⁰-Orn²¹-Leu²²-Leu²³-Gln²⁴-Asp²⁵-Ile²⁶-Nle²⁷-D-Arg²⁸-Har²⁹-R²-NH₂,
wherein R¹ is PhAc-Ada or PhAc;
A⁶ is Phe(F)₅ or Cpa;
A⁸ is Ala or Me-Ala;
A¹⁰ is Tyr(Me) or FPa5;
A¹⁷ is Leu or Glu; and
R² is Ada, Agm, or absent.

34. The method of claim 32, wherein the GHRH peptide antagonist is selected from the group consisting of:
Phac-Ada-Tyr¹-D-Arg²-Asp³-Ala⁴-Ile⁵-Phe(F)₅⁶-Thr⁷-Ala⁸-Har⁹-Tyr(me)¹⁰-His¹¹-Orn¹²-Val¹³-Leu¹⁴-Abu¹⁵-Glu¹⁶-Leu¹⁷-Ser¹⁸-Ala¹⁹-His²⁰-Orn²¹-Leu²²-Leu²³-Glu²⁴-Asp²⁵-Ile²⁶-Nle²⁷-D-Arg²⁸-Har²⁹-NH₂ (MIA-602),
Phac-Ada-Tyr¹-D-Arg²-Asp³-Ala⁴-Ile⁵-Phe(F)₅⁶-Thr⁷-Ala⁸-Har⁹-Tyr(me)¹⁰-His¹¹-Orn¹²-Val¹³-Leu¹⁴-Abu¹⁵-Glu¹⁶-Leu¹⁷-Ser¹⁸-Ala¹⁹-His²⁰-Orn²¹-Leu²²-Leu²³-Glu²⁴-Asp²⁵-Ile²⁶-Nle²⁷-D-Arg²⁸-Har²⁹-Agm-NH₂ (MIA-604),
Phac-Ada-Tyre-D-Arg²-Asp³-Ala⁴-Ile⁵-Phe(F)₅⁶-Thr⁷-Me-Ala⁸-Har⁹-Tyr(Me)¹⁰-His¹¹-Orn¹²-Val¹³-Leu¹⁴-Abu¹⁵-Glu¹⁶-Leu¹⁷-Ser¹⁸-Ala¹⁹-His²⁰-Orn²¹-Leu²²-Leu²³-Glu²⁴-Asp²⁵-Ile²⁶-Nle²⁷-D-Arg²⁸-Har²⁹-Agm-NH₂ (MIA-606),
Phac-Tyr¹-D-Arg²-Asp³-Ala⁴-Ile⁵-Cpa⁶-Thr⁷-Ala⁸-Har⁹-Fpa5¹⁰-His¹¹-Orn¹²-Val¹³-Leu¹⁴-Abu¹⁵-Glu¹⁶-Leu¹⁷-Ser¹⁸-Ala¹⁹-His²⁰-Orn²¹-Leu²²-Leu²³-Glu²⁴-Asp²⁵-Ile²⁶-Nle²⁷-D-Arg²⁸-Har²⁹-Ada-NH₂ (MIA-610),
Phac-Ada-Tyr¹-D-Arg²-Asp³-Ala⁴-Ile⁵-Cpa⁶-Thr⁷-Ala⁸-Har⁹-Fpa5¹⁰-His¹¹-Orn¹²-Val¹³-Leu¹⁴-Abu¹⁵-Glu¹⁶-Leu¹⁷-Ser¹⁸-Ala¹⁹-His²⁰-Orn²¹-Leu²²-Leu²³-Glu²⁴-Asp²⁵-Ile²⁶-Nle²⁷-D-Arg²⁸-Har²⁹-Ada-NH₂ (MIA-640), and
Phac-Ada-Tyr¹-D-Arg²-Asp³-Ala⁴-Ile⁵-Cpa⁶-Thr⁷-Ala⁸-Har⁹-Fpa5¹⁰-His¹¹-Orn¹²-Val¹³-Leu¹⁴-Abu¹⁵-Gln¹⁶-Leu¹⁷-Ser¹⁸-Ala¹⁹-His²⁰-Orn²¹-Leu²²-Leu²³-Gln²⁴-Asp²⁵-Ile²⁶-Nle²⁷-D-Arg²⁸-Har²⁹-NH₂ (MIA-690).

35. The method of claim 32, wherein the GHRH peptide antagonist has the following amino acid sequence:
Phac-Ada-Tyr¹-D-Arg²-Asp³-Ala⁴-Ile⁵-Cpa⁶-Thr⁷-Ala⁸-Har⁹-Fpa5¹⁰-His¹¹-Orn¹²-Val¹³-Leu¹⁴-Abu¹⁵-Gln¹⁶-Leu¹⁷-Ser¹⁸-Ala¹⁹-His²⁰-Orn²¹-Leu²²-Leu²³-Gln²⁴-Asp²⁵-Ile²⁶-Nle²⁷-D-Arg²⁸-Har²⁹-NH₂ (MIA-690).

36. The method of claim 32, wherein the neurodegenerative disease is selected from Alzheimer's disease, senile dementia, dementia with Lewy Bodies, Parkinson's disease, Huntington's disease, amyotrophic lateral sclerosis, and combination thereof.

37. The method of claim 32, wherein the GHRH peptide antagonist administration is at a dosage of about 0.005 mg/kg/dose to about 100 mg/kg/dose.

38. The method of claim 32, further comprising administering the GHRH peptide antagonist with at least one therapeutic agent selected from the group consisting of a NMDA receptor antagonist, an inhibitor of amyloid AB peptide, a phosphodiesterase (PDE5) inhibitor, a PDE4 inhibitor, a monoamine oxidase inhibitor, a VEGF protein, a trophic growth factor, a HIF activator, a HIF prolyl A-hydroxylases inhibitor, an anti-apoptotic compound, an activity-dependent neurotrophic protein (ADNP) agonist, an activity-dependent neurotrophic factor (ADNF) agonist, an activator of an AMPA-type glutamate receptor, a serotonin 5-HT1A receptor agonist, a serotonin IA receptor antagonist, a nicotinic alpha-7 receptor agonist, a neuronal L-type calcium channel modulator, a 5-HT4 receptor agonist, an anti-inflammatory agent, a pharmaceutically acceptable salt thereof and combinations thereof.

39. The method of claim 38, wherein the therapeutic agent is co-administered, concurrently administered, or sequentially administered with the GHRH peptide antagonist.

40. The method of claim 32, wherein the administration of the GHRH peptide antagonist is parenteral administration selected from the group consisting of subcutaneous, intramuscular, intraperitoneal, intracavity, intrathecal, transdermal, and intravenous injection.

41. A method for treating a subject having amyloid plaque deposits comprising administering to said subject a therapeutic amount of growth hormone releasing hormone (GHRH) peptide antagonist having the amino acid sequence (formula I):
R¹-Tyr¹-D-Arg²-Asp³-A⁴-Ile⁵-A⁶-Thr⁷-A⁸-Har⁹-A¹⁰-A¹¹-A¹²-Val¹³-Leu¹⁴-A¹⁵-Gln¹⁶-A¹⁷-Ser¹⁸-Ala¹⁹-A²⁰-A²¹-Leu²²-Leu²³-Gln²⁴-Asp²⁵Ile²⁶-Nle²⁷-D-Arg²⁸-A²⁹-R²-R³-NH₂,
wherein R¹ is PhAc, Nac, Oct, N-Me-Aib, Dca, Ac-Ada, Fer, Ac-Amc, Me-NH-Sub, PhAc-Ada, Ac-Ada-D-Phe, Ac-Ada-Phe, Dca-Ada, Nac, Nac-Ada, Ada-Ada, or CH₃(CH₂)₁₀-CO-Ada;
A⁴ is Ala or Me-Ala;
A⁶ is Cpa or Phe(F)₅;
A⁸ is Ala, Pal, Dip, or Me-Ala;
A¹⁰ is FPa5,Tyr(Alk) where Alk is Me or Et;
A¹¹ is His or Arg;
A¹² is Lys, Lys(0-11), Lys(Me)₂, or Orn;
A¹⁵ is Abu or Orn;
A¹⁷ is Leu or Glu;
A²⁰ is Har or His;
A²¹ is Lys, Lys(Me)₂ or Orn;
A²⁹ is Har, Arg or Agm;
R² is β-Ala, Amc, Apa, Ada, AE₂A, AE₄P, ε-Lys(α-NH₂), Agm, or absent; and
R³ is Lys(Oct), Ahx, or absent.

42. The method of claim 41, wherein the GHRH peptide antagonist has the amino acid sequence (formula II):
R¹-Tyr¹-D-Arg²-Asp³-Ala⁴-Ile⁵-A⁶-Thr⁷-A⁸-Har⁹-A¹⁰-His¹¹-Orn¹²-Val¹³-Leu¹⁴-Abu¹⁵-Gln¹⁶-A¹⁷-Ser¹⁸-Ala¹⁹-His²⁰-Orn²¹-Leu²²-Leu²³-Gln²⁴-Asp²⁵-Ile²⁶-Nle²⁷-D-Arg²⁸-Har²⁹-R²-NH₂,
wherein R¹ is PhAc-Ada or PhAc;
A⁶ is Phe(F)₅ or Cpa;
A⁸ is Ala or Me-Ala;
A¹⁰ is Tyr(Me) or FPa5;
A¹⁷ is Leu or Glu; and
R² is Ada, Agm, or absent.

43. The method of claim 41, wherein the GHRH peptide antagonist is selected from the group consisting of:
Phac-Ada-Tyr¹-D-Arg²-Asp³-Ala⁴-Ile⁵-Phe(F)₅⁶-Thr⁷-Ala⁸-Har⁹-Tyr(Me)¹⁰-His¹¹-Orn¹²-Val¹³-Leu¹⁴-Abu¹⁵-Gln¹⁶-Leu¹⁷-Ser¹⁸-Ala¹⁹-His²⁰-Orn²¹-Leu²²-Leu²³-Gln²⁴-Asp²⁵-Ile²⁶-Nle²⁷-D-Arg²⁸-Har²⁹-NH₂ (MIA-602),
Phac-Ada-Tyr¹-D-Arg²-Asp³-Ala⁴-Ile⁵-Phe(F)₅⁶-Thr⁷-Ala⁸-Har⁹-Tyr(Me)¹⁰-His¹¹-Orn¹²-Val¹³-Leu¹⁴-Abu¹⁵-Gln¹⁶-Leu¹⁷-Ser¹⁸-Ala¹⁹-His²⁰-Orn²¹-Leu²²-Leu²³-Gln²⁴-Asp²⁵-Ile²⁶-Nle²⁷-D-Arg²⁸-Har²⁹-Agm-NH₂ (MIA-604),
Phac-Ada-Tyr¹-D-Arg²-Asp³-Ala⁴-Ile⁵-Phe(F)₅⁶-Thr⁷-Me-Ala⁸-Har⁹-Tyr(Me)¹⁰-His¹¹-Orn¹²-Val¹³-Leu¹⁴-Abu¹⁵-Gln¹⁶-Leu¹⁷-Ser¹⁸-Ala¹⁹-His²⁰-Orn²¹-Leu²²-Leu²³-Gln²⁴-Asp²⁵-Ile²⁶-Nle²⁷-D-Arg²⁸-Har²⁹-Agm-NH₂ (MIA-606),
Phac-Tyr¹-D-Arg²-Asp³-Ala⁴-Ile⁵-Cpa⁶-Thr⁷-Ala⁸-Har⁹-Fpa5¹⁰-His¹¹-Orn¹²-Val¹³-Leu¹⁴-Abu¹⁵-Gln¹⁶-Leu¹⁷-Ser¹⁸-Ala¹⁹-His²⁰-Orn²¹-Leu²²-Leu²³-Gln²⁴-Asp²⁵-Ile²⁶-Nle²⁷-D-Arg²⁸-Har²⁹-Ada-NH₂ (MIA-610),
Phac-Ada-Tyr¹-D-Arg²-Asp³-Ala⁴-Ile⁵-Cpa⁶-Thr⁷-Ala⁸-Har⁹-Fpa5¹⁰-His¹¹-Orn¹²-Val¹³-Leu¹⁴-Abu¹⁵-Gln¹⁶-Glu¹⁷-Ser¹⁸-Ala¹⁹-His²⁰-Orn²¹-Leu²²-Leu²³-Gln²⁴-Asp²⁵-Ile²⁶-Nle²⁷-D-Arg²⁸-Har²⁹-Ada-NH₂ (MIA-640), and
Phac-Ada-Tyr¹-D-Arg²-Asp³-Ala⁴-Ile⁵-Cpa⁶-Thr⁷-Ala⁸-Har⁹-Fpa5¹⁰-His¹¹-Orn¹²-Val¹³-Leu¹⁴-Abu¹⁵-Gln¹⁶-Leu¹⁷-Ser¹⁸-Ala¹⁹-His²⁰-Orn²¹-Leu²²-Leu²³-Gln²⁴-Asp²⁵-Ile²⁶-Nle²⁷-D-Arg²⁸-Har²⁹-NH₂ (MIA-690).

44. The method of claim 41, wherein the amyloid plaque deposits are associated with a disease selected from the group consisting of Mediterranean fever, Muckle-Wells syndrome, idiopathic myeloma, amyloid polyneuropathy, amyloid cardiomyopathy, systemic neuritic amyloidosis, amyloid polyneuropathy, hereditary cerebral hemorrhage with amyloidosis, Down's syndrome, Scrapie, Creutzfeldt-Jacob disease, Kuru, Gerstamnn-Straussler-Scheinker syndrome, medullary carcinoma of the thyroid, isolated atrial amyloid, β2-microglobulin amyloid in dialysis patients, inclusion body myositis, β2-amyloid deposits in muscle wasting disease, type II diabetes, and combinations thereof.

45. The method of claim 41, wherein the administration of the GHRH peptide antagonist is parenteral administration selected from subcutaneous, intramuscular, intraperitoneal, intracavity, intrathecal, transdermal, and intravenous injection.

46. The method of claim 41, wherein the GHRH peptide antagonist administration is at a dosage of about 0.005 mg/kg/dose to about 100 mg/kg/dose.

47. The method of claim 41, further comprising administering the GHRH peptide antagonist with at least one therapeutic agent selected from the group consisting of a NMDA receptor antagonist, an inhibitor of amyloid AB peptide, a phosphodiesterase (PDE5) inhibitor, a PDE4 inhibitor, a monoamine oxidase inhibitor, a VEGF protein, a trophic growth factor, a HIF activator, a HIF prolyl A-hydroxylases inhibitor, an anti-apoptotic compound, an activity-dependent neurotrophic protein (ADNP) agonist, an activity-dependent neurotrophic factor (ADNF) agonist, an activator of an AMPA-type glutamate receptor, a serotonin 5-HT1A receptor agonist, a serotonin IA receptor antagonist, a nicotinic alpha-7 receptor agonist, a neuronal L-type calcium channel modulator, a 5-HT4 receptor agonist, an anti-inflammatory agent, and a pharmaceutically acceptable salt thereof.

48. A method of protecting neuronal cells from oxidative stress comprising contacting the neuronal cells with a growth hormone releasing hormone (GHRH) peptide antagonist having the amino acid sequence (formula I):
R¹-Tyr¹-D-Arg²-Asp³-A⁴-Ile⁵-A⁶-Thr⁷-A⁸-Har⁹-A¹⁰-A¹¹-A¹²-Val¹³-Leu¹⁴-A¹⁵-Gln¹⁶-A¹⁷-Ser¹⁸-Ala¹⁹-A²⁰-A²¹-Leu²²-Leu²³-Gln²⁴-Asp²⁵-Ile²⁶-Nle²⁷-D-Arg²⁸-A²⁹-R²-R³-NH₂,
wherein R¹ is PhAc, Nac, Oct, N-Me-Aib, Dca, Ac-Ada, Fer, Ac-Amc, Me-NH-Sub, PhAc-Ada, Ac-Ada-D-Phe, Ac-Ada-Phe, Dca-Ada, Nac, Nac-Ada, Ada-Ada, or CH₃(CH₂)₁₀-CO-Ada;
A⁴ is Ala or Me-Ala;
A⁶ is Cpa or Phe(F)₅;
A⁸ is Ala, Pal, Dip, or Me-Ala;
A¹⁰ is FPa5,Tyr(Alk) where Alk is Me or Et;
A¹¹ is His or Arg;
A¹² is Lys, Lys(0-1 l), Lys(Me)₂, or Orn;
A¹⁵ is Abu or Orn;
A¹⁷ is Leu or Glu;
A²⁰ is Har or His;
A²¹ is Lys, Lys(Me)₂ or Orn;
A²⁹ is Har, Arg or Agm;
R² is β-Ala, Amc, Apa, Ada, AE₂A, AE₄P, ε-Lys(α-NH₂), Agm, or absent; and
R³ is Lys(Oct), Ahx, or absent.

49. The method of claim 48, wherein the GHRH peptide antagonist has the amino acid sequence (formula II):
R¹-Tyr¹-D-Arg²-Asp³-Ala⁴-Ile⁵-A⁶-Thr⁷-A⁸-Har⁹-A¹⁰-His¹¹-Orn¹²-Val¹³-Leu¹⁴-Abu¹⁵-Gln¹⁶-A¹⁷-Ser¹⁸-Ala¹⁹-His²⁰-Orn²¹-Leu²²-Leu²³-Gln²⁴-Asp²⁵-Ile²⁶-Nle²⁷-D-Arg²⁸-Har²⁹-R²-NH₂,
wherein R¹ is PhAc-Ada or PhAc;
A⁶ is Phe(F)₅ or Cpa;
A⁸ is Ala or Me-Ala;
A¹⁰ is Tyr(Me) or FPa5;
A¹⁷ is Leu or Glu; and
R² is Ada, Agm, or absent.

50. The method of claim 48, wherein the GHRH peptide antagonist is selected from the group consisting of:
Phac-Ada-Tyr¹-D-Arg²-Asp³-Ala⁴-Ile⁵-Phe(F)⁵⁶-Thr⁷-Ala⁸-Har⁹-Tyr(Me)¹⁰-His¹¹-Orn¹²-Val¹³-Leu¹⁴-Abu¹⁵-Gln¹⁶-Leu¹⁷-Ser¹⁸-Ala¹⁹-His²⁰-Orn²¹-Leu²²-Leu²³-Gln²⁴-Asp²⁵-Ile²⁶-Nle²⁷-D-Arg²⁸-Har²⁹-NH₂ (MIA-602),
Phac-Ada-Tyr¹-D-Arg²-Asp³-Ala⁴-Ile⁵-Phe(F)₅⁶-Thr⁷-Ala⁸-Har⁹-Tyr(Me)¹⁰-His¹¹-Orn¹²-Val¹³-Leu¹⁴-Abu¹⁵-Gln¹⁶-Leu¹⁷-Ser¹⁸-Ala¹⁹-His²⁰-Orn²¹-Leu²²-Leu²³-Gln²⁴-Asp²⁵-Ile²⁶-Nle²⁷-D-Arg²⁸-Har²⁹-Agm-NH₂ (MIA-604),
Phac-Ada-Tyr¹-D-Arg²-Asp³-Ala⁴-Ile⁵-Phe(F)₅⁶-Thr⁷-Me-Ala⁸-Har⁹-Tyr(Me)¹⁰-His¹¹-Orn¹²-Val¹³-Leu¹⁴-Abu¹⁵-Gln¹⁶-Leu¹⁷-Ser¹⁸-Ala¹⁹-His²⁰-Orn²¹-Leu²²-Leu²³-Gln²⁴-Asp²⁵-Ile²⁶-Nle²⁷-D-Arg²⁸-Har²⁹-Agm-NH₂ (MIA-606),
Phac-Tyr¹-D-Arg²-Asp³-Ala⁴-Ile⁵-Cpa⁶-Thr⁷-Ala⁸-Har⁹-Fpa5¹⁰-His¹¹-Orn¹²-Val¹³-Leu¹⁴-Abu¹⁵-Gln¹⁶-Leu¹⁷-Ser¹⁸-Ala¹⁹-His²⁰-Orn²¹-Leu²²-Leu²³-Gln²⁴-Asp²⁵-Ile²⁶-Nle²⁷-D-Arg²⁸-Har²⁹-Ada-NH₂ (MIA-610),
Phac-Ada-Tyr¹-D-Arg²-Asp³-Ala⁴-Ile⁵-Cpa⁶-Thr⁷-Ala⁸-Har⁹-Fpa5¹⁰-His¹¹-Orn¹²-Val¹³-Leu¹⁴-Abu¹⁵-Gln¹⁶-Glu¹⁷-Ser¹⁸-Ala¹⁹-His²⁰-Orn²¹-Leu²²-Leu²³-Gln²⁴-Asp²⁵-Ile²⁶-Nle²⁷-D-Arg²⁸-Har²⁹-Ada-NH₂ (MIA-640), and
Phac-Ada-Tyr^{e}-D-Arg²-Asp³-Ala⁴-Ile⁵-Cpa⁶-Thr⁷-Ala⁸-Har⁹-Fpa5¹⁰-His¹¹-Orn¹²-Val¹³-Leu¹⁴-Abu¹⁵-Gln¹⁶-Leu¹⁷-Ser¹⁸-Ala¹⁹-His²⁰-Orn²¹-Leu²²-Leu²³-Gln²⁴-Asp²⁵-Ile²⁶-Nle²⁷-D-Arg²⁸-Har²⁹-NH₂ (MIA-690).

51. The method of claim 48, wherein the local concentration of the GHRH peptide antagonist used is from about 1 nM to about 100 mM.
